SEQUENCE LISTING (55 sequences)

SEQ ID NO: 1 - DNA, 1040 bp, Trichoderma reesei
  exon (1)..(574)
  Intron (575)..(651)
  exon (652)..(1037)
atggatggcatatcgaatctgctggccatgctcaacgtgagcgacctgacgccggcagaatggtgccaaatcttcttcgtcgccaacgctggcttcatcctgatgctgcaagctctcccggacgacatgcgtcgggcgttgatggactacggcgccagaagagccacgtcccgccaggcttccgcaaaaaagggtcaggccgaggtacccaaggaaggcttcttcgacgtcttgacgagctatggccaggtgccgcactcgtggttcatacacttctacatcacgtccgtgtcgtggtccatcttctggggctggcagttcgtgtccaagggatcggtgatgcgggccctggccgagaggcagcacagagctgcggggaatgagccatcgtcggaggtcgagctcacagcaacgctggtcgcgtggctcttgatgagctcgcagggagcgagaagactgtttgagtgtctctttgtcaccaagcctgggtcatcgcccatgtggtttgtccattgggttctcggattggcctattacacaaccctggggatctctgtctggattcaaggctcaggttcgtctcacacggctattaacaaaagagtctagtttcgtggctgtcttggtcgtgacgctgacgtgagttcacaggagcaattcttcggtcgtgggagtccccgcagcccatccacttcacgccacaaatcattatcggggcggcagtctttggattcgcaggcgcccagcaaaatgaatgccacaggtatctggcagggctcaagaagtacacgctcccgagcgagggctggttcaagtacttcgtctgcccccactacacattcgaatgcctcctctacttgagccttgccgttgttgcggcgccgacaggatgctgggtgaacggatccatcctgtgctcattgacgtttgtcctggccaatgtcggctcaacggcatacggcaccaagatttggtatgccgaaaagttcggggcggacaaggtggaggggaaatggatcatgattccctttgtattctaa

SEQ ID NO: 2 - DNA, 963 bp, Trichoderma reesei
  CDS (1)..(960)
atggatggcatatcgaatctgctggccatgctcaacgtgagcgacctgacgccggcagaatggtgccaaatcttcttcgtcgccaacgctggcttcatcctgatgctgcaagctctcccggacgacatgcgtcgggcgttgatggactacggcgccagaagagccacgtcccgccaggcttccgcaaaaaagggtcaggccgaggtacccaaggaaggcttcttcgacgtcttgacgagctatggccaggtgccgcactcgtggttcatacacttctacatcacgtccgtgtcgtggtccatcttctggggctggcagttcgtgtccaagggatcggtgatgcgggccctggccgagaggcagcacagagctgcggggaatgagccatcgtcggaggtcgagctcacagcaacgctggtcgcgtggctcttgatgagctcgcagggagcgagaagactgtttgagtgtctctttgtcaccaagcctgggtcatcgcccatgtggtttgtccattgggttctcggattggcctattacacaaccctggggatctctgtctggattcaaggctcaggagcaattcttcggtcgtgggagtccccgcagcccatccacttcacgccacaaatcattatcggggcggcagtctttggattcgcaggcgcccagcaaaatgaatgccacaggtatctggcagggctcaagaagtacacgctcccgagcgagggctggttcaagtacttcgtctgcccccactacacattcgaatgcctcctctacttgagccttgccgttgttgcggcgccgacaggatgctgggtgaacggatccatcctgtgctcattgacgtttgtcctggccaatgtcggctcaacggcatacggcaccaagatttggtatgccgaaaagttcggggcggacaaggtggaggggaaatggatcatgattccctttgtattctaa

SEQ ID NO: 3 - protein, 320 aa, Trichoderma reesei
MDGISNLLAMLNVSDLTPAEWCQIFFVANAGFILMLQALPDDMRRALMDYGARRATSRQASAKKGQAEVPKEGFFDVLTSYGQVPHSWFIHFYITSVSWSIFWGWQFVSKGSVMRALAERQHRAAGNEPSSEVELTATLVAWLLMSSQGARRLFECLFVTKPGSSPMWFVHWVLGLAYYTTLGISVWIQGSGAILRSWESPQPIHFTPQIIIGAAVFGFAGAQQNECHRYLAGLKKYTLPSEGWFKYFVCPHYTFECLLYLSLAVVAAPTGCWVNGSILCSLTFVLANVGSTAYGTKIWYAEKFGADKVEGKWIMIPFVF

SEQ ID NO: 4 - DNA, 1123 bp, Aspergillus niger
  exon (1)..(99)
  Intron (100)..(189)
  exon (190)..(670)
  Intron (671)..(722)
  exon (723)..(1120)
atggagctccccttcagcctggtggatacagctctttcctccacgcacattgacgccatcgacgcccttcgagcctttttccttttggcatcctgcacggtatgcaccgatatgttgccatcccaaattcgaggcggacgacgttgcaaagtgaccctgcatcaactaaacccagaatatcccaactagatactctccgtcagcatcctgccagattcgcttcattcacgcttcattccttatggcgctcgtacaacttcgacggccgactcgcagtcctcgcagtcctcgcccccggcgtcctcggactcctccccagccgcccgcgctcttgactacgctgccgccttgacagtccctcacagctacttcacgcaattctacattgcctccgttttggcctcggtcttctgggctgcgcagcttctgtgtcaaggagccgtgttccaggcctttgcaacgagaatcagtccggaacatatgcaacagtccatgtccatacaccaagtcttcttgtgctgggcgctcatgctcattcagggtgcaaggcggctatatgagtgcaaggccttcgccaagccttcagcctcaagaatgtggtttgttcactggctcgtaggccttgcattttacctagcagtatccgttgccttatggatcgaaggagcaggtaccactctgactgacaggctaaaataccgaaccccactgattgcatctaggagccctgctgtctcataaagtgattctcgacgacctcaaggttacaatagcaccctctctccgcacctttgtctgcattcctcttttcttgattgcatcaggcatccaacacgactgccaccattacctcttctctttgaagaaatataccctgcccactcaccccatctttcgcaggatcgtatgtccacattatacggcagagtgtgtgatatatctttcgttggctctgctgggcgcgcccaagggcgaggtcgtaaacaagtcgttgttgtccgctctcgtatttgttgttatcaacttgggcgtcaccgctgcgaacactaggcattggtatatgcgaaaattcggcgaggagtctgtgcgggaaagatcgaacatgatcccttgggtttactaa

SEQ ID NO: 5 - DNA, 981 bp, Aspergillus niger
  CDS (1)..(978)
atggagctccccttcagcctggtggatacagctctttcctccacgcacattgacgccatcgacgcccttcgagcctttttccttttggcatcctgcacgatactctccgtcagcatcctgccagattcgcttcattcacgcttcattccttatggcgctcgtacaacttcgacggccgactcgcagtcctcgcagtcctcgcccccggcgtcctcggactcctccccagccgcccgcgctcttgactacgctgccgccttgacagtccctcacagctacttcacgcaattctacattgcctccgttttggcctcggtcttctgggctgcgcagcttctgtgtcaaggagccgtgttccaggcctttgcaacgagaatcagtccggaacatatgcaacagtccatgtccatacaccaagtcttcttgtgctgggcgctcatgctcattcagggtgcaaggcggctatatgagtgcaaggccttcgccaagccttcagcctcaagaatgtggtttgttcactggctcgtaggccttgcattttacctagcagtatccgttgccttatggatcgaaggagcaggagccctgctgtctcataaagtgattctcgacgacctcaaggttacaatagcaccctctctccgcacctttgtctgcattcctcttttcttgattgcatcaggcatccaacacgactgccaccattacctcttctctttgaagaaatataccctgcccactcaccccatctttcgcaggatcgtatgtccacattatacggcagagtgtgtgatatatctttcgttggctctgctgggcgcgcccaagggcgaggtcgtaaacaagtcgttgttgtccgctctcgtatttgttgttatcaacttgggcgtcaccgctgcgaacactaggcattggtatatgcgaaaattcggcgaggagtctgtgcgggaaagatcgaacatgatcccttgggtttactaa

SEQ ID NO: 6 - protein, 326 aa, Aspergillus niger
MELPFSLVDTALSSTHIDAIDALRAFFLLASCTILSVSILPDSLHSRFIPYGARTTSTADSQSSQSSPPASSDSSPAARALDYAAALTVPHSYFTQFYIASVLASVFWAAQLLCQGAVFQAFATRISPEHMQQSMSIHQVFLCWALMLIQGARRLYECKAFAKPSASRMWFVHWLVGLAFYLAVSVALWIEGAGALLSHKVILDDLKVTIAPSLRTFVCIPLFLIASGIQHDCHHYLFSLKKYTLPTHPIFRRIVCPHYTAECVIYLSLALLGAPKGEVVNKSLLSALVFVVINLGVTAANTRHWYMRKFGEESVRERSNMIPWVY

SEQ ID NO: 7 - DNA, 1152 bp, Aspergillus oryzae
  exon (1)..(105)
  Intron (106)..(216)
  exon (217)..(685)
  Intron (686)..(751)
  exon (752)..(1149)
atgaccatggactttcttcccagtctcgtcgactgggcactggcgactgcccacatggacgcggtggatgtccttcgagctttctttctctttgcatcgtgtacggtatgaaaacatgttgacaatgacgtagtggcagaacgccgagcatcatacggcatcagtctaccaaatatttttttctatctaaaatggcttcaaaactaaactccccagatcctgtcggtcagcctgttggattcccttcgttcgcgcttcgtcccttatggcgctcgtgcaaccgtgacggccgagtcggatactaccccgtctgagccttcgagcagctcccccttaactcacattctcgactatcttgcctctttgaaagttccacacagctatttcacacagttctacgtcgtctccctgctatcctcggttttctgggcccttcagcttatgtgccatggacaagcgttccaagccatcgcgacgagggttcactcggaacacctacagaggacgatgtcgatcaaccagattatgttgtgctgggtactgatgctcgcgcaaggtgtaagacggttacatgagtgcttcaccttttccaagccttcttcttcccagatgtggtttgtccactggctagcaggcattgctttctatctagcagtctcaatcgccctgtggattgaggggacaggtatgcagttcccgtggccaagcacgattgctgtcccgctaatttcgatcggttcgaaattcacagaaacgctgttgtcccataaattgagcctcgacgatgtaaccgtgaacaacgccccaagccttcgtacatttctctgcttgccaatctttctgtttgcatctggcctacaacatgacgcccaccactatcttttctctctcaagaagtacacccttccctcccaccccttgttccgcagcatcgtatgtccgcactacacggctgaatgtgcgatctacctatcattggccctgcttgccgctccccggggagagatgatcaataagacagttttgtccgctgccgtctttgtcacggtcaatttaggcgtcactgcgtccgagaccaaacgctggtacatgcagaaattcggcgagagctcggtgcgggagaggtggaatatgatcccttgggtatactaa

SEQ ID NO: 8 - DNA, 975 bp, Aspergillus oryzae
  CDS (1)..(972)
atgaccatggactttcttcccagtctcgtcgactgggcactggcgactgcccacatggacgcggtggatgtccttcgagctttctttctctttgcatcgtgtacgatcctgtcggtcagcctgttggattcccttcgttcgcgcttcgtcccttatggcgctcgtgcaaccgtgacggccgagtcggatactaccccgtctgagccttcgagcagctcccccttaactcacattctcgactatcttgcctctttgaaagttccacacagctatttcacacagttctacgtcgtctccctgctatcctcggttttctgggcccttcagcttatgtgccatggacaagcgttccaagccatcgcgacgagggttcactcggaacacctacagaggacgatgtcgatcaaccagattatgttgtgctgggtactgatgctcgcgcaaggtgtaagacggttacatgagtgcttcaccttttccaagccttcttcttcccagatgtggtttgtccactggctagcaggcattgctttctatctagcagtctcaatcgccctgtggattgaggggacagaaacgctgttgtcccataaattgagcctcgacgatgtaaccgtgaacaacgccccaagccttcgtacatttctctgcttgccaatctttctgtttgcatctggcctacaacatgacgcccaccactatcttttctctctcaagaagtacacccttccctcccaccccttgttccgcagcatcgtatgtccgcactacacggctgaatgtgcgatctacctatcattggccctgcttgccgctccccggggagagatgatcaataagacagttttgtccgctgccgtctttgtcacggtcaatttaggcgtcactgcgtccgagaccaaacgctggtacatgcagaaattcggcgagagctcggtgcgggagaggtggaatatgatcccttgggtatactaa

SEQ ID NO: 9 - protein, 324 aa, Aspergillus oryzae
MTMDFLPSLVDWALATAHMDAVDVLRAFFLFASCTILSVSLLDSLRSRFVPYGARATVTAESDTTPSEPSSSSPLTHILDYLASLKVPHSYFTQFYVVSLLSSVFWALQLMCHGQAFQAIATRVHSEHLQRTMSINQIMLCWVLMLAQGVRRLHECFTFSKPSSSQMWFVHWLAGIAFYLAVSIALWIEGTETLLSHKLSLDDVTVNNAPSLRTFLCLPIFLFASGLQHDAHHYLFSLKKYTLPSHPLFRSIVCPHYTAECAIYLSLALLAAPRGEMINKTVLSAAVFVTVNLGVTASETKRWYMQKFGESSVRERWNMIPWVY

SEQ ID NO: 10 - DNA, 1014 bp, Fusarium venenatum
  exon (1)..(571)
  Intron (572)..(622)
  exon (623)..(1011)
atggaagaattgatacaccaatccatcgatctgatgtcgattttgacaccagcacaatggtgtcagactttcttcgccctttctacggccatcgtccttggtatccaagccctcccacaagatgttcgcagcgctctcatggactacggcgcccgaagacccaaagatgcgaaacacggaaaggagcaagaagaaaacggacagaaagctctcgtgcctctgaggtcatttatgaagaatttaacagagtacgggcaagtgccccattcatggttcttgcacttttacattgtttcggtggccctgtcgggtttctgggcgtggcagtatctcactcaaggtcatgtactgaaaagcattgtgacgtggcagaatcgagctgatggaccgtccatgagcctggagcaaattttcgtggcatggttgctcatggcgttgcagggttcgagaaggctttacgagagtttgtttgtgttcaagccagggtcatcacctatgtggtttattcattgggcacttggtctttcgtactacattgcaatgagccttgctgtctgggtcgagggttccagtatgttttcaatattcacacctggctacaacagaagctgacaagttgcaggcgccattttagcagcctgggattctccctatcaacctctccgggttccacgacggctaccttcagccctagcgttatatttcgtggcttacttcaagcaaaaccaatgtcacagacacttggcaagcctcaagaagtataccctcccaagtgagggatggttcaagtatataatctgtccacactatactttagaatgccttgtgtaccttgccattgcatggatcgctgcaccacacggccaaatcttcaacaaaagtatcttgggcgctgtcatgtttgtggctgtgaatctcggtgccacggcaaagggcaccaaggtatggtatgagcagaaattcggtgcaaacaaggtcgctgggcggtggctcatgatcccgcctgtctactag

SEQ ID NO: 11 - DNA, 11526 bp, Artificial sequence
  Plasmid pNJOC577
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcgtttaaacgtgctggacctgctaaggaggaagttttttttgggacggaaaagcctggtgaggcatgaagatggagagagttttcggtgtgtgaggatttcgagtatcatggaaaaagaaggaattcaagtttgtaacagaaagatgtttaatggttgatttgcgaacatgatagacgtccaaatttcataatcttttgcaattgagccttggagatattacgaaaatgagatgtgagatctcgaaatgttgaggaatattcatgcctggttacacctgccgtcgttgtcatgccactcgggcgggcaggagcatccatgcttcgcctagcaccaaggattggcaaccgccagatggatcatgacggcaggattccttgaatcggttctagacctttttgtgcacgtgccttagctataagccaacgacttgcaaagcggaaggcagcacctgggcatggctgggaaagaagcttgaagcatgcgccttgctgttgccttcgcttggttgatgttatcggggcaacttgcttgagacattggtcgcacttgccgacctgtgaataggcaagacatatttcgtcagtagtacctacccagcgtaagggatttcacgaggaagaaggtgagtcatcgggttcaaatgctgtactacctcttgatggagttgcagatactatcacgagcttgtgtgctactattctgttggaattcggtctcttataccgtccactcggttcagcactgtacgaacaacgtcttcatgctgcaacactggtccatacatcctttgcatccgtcggtcaatcctgcatcgcctcataccaagtccctcctcgtggaaccaagccctctcatcctccttgtctcccagctcttctcaagccatcaaatcccaactgcaacatggaacttgaagttgctgtgaagcaagcgccaatacgacgaatccaccgcccaatcaaaaccccacagcagtgcaacgccacctcagcgtcaattagactacggattggccgtcaagccgtgtatcgtattgctggttgccaagagcctcagtcacaagttcaaaaagcaagcaaatacgcattctctctccttccagtggcaatccatgatttcattgaagccatgttctggtagtaggatctatgcgagctgcagagaagtttctccgcactccatatctgctgcaatggccggcaatcaagtaaatgcaccgagagtcgttggttcaagggacggagcaatgtgccgattttgggaccacaagagcggcggctcagcatgcaggcgaccaaatttcgcacaattgtgcttctccttggtaggggcatatcactccacgcctggctttggcagtgcaattcggcagctttcaaccagcgcaacgaatgtctgccctttcatcccttcctgagtcattttctcccattcactttatctggcccctcaatctctaaggtcttgttcttgtggcctggctgaaccttgcctcgccgcatcacgccagccgcttgaattcacaacatggatggcatatcgaatctgctggccatgctcaacgtgagcgacctgacgccggcagaatggtgccaaatcttcttcgtcgccaacgctggcttcatcctgatgctgcaagctctcccggacgacatgcgtcgggcgttgatggactacggcgccagaagagccacgtcccgccaggcttccgcaaaaaagggtcaggccgaggtacccaaggaaggcttcttcgacgtcttgacgagctatggccaggtgccgcactcgtggttcatacacttctacatcacgtccgtgtcgtggtccatcttctggggctggcagttcgtgtccaagggatcggtgatgcgggccctggccgagaggcagcacagagctgcggggaatgagccatcgtcggaggtcgagctcacagcaacgctggtcgcgtggctcttgatgagctcgcagggagcgagaagactgtttgagtgtctctttgtcaccaagcctgggtcatcgcccatgtggtttgtccattgggttctcggattggcctattacacaaccctggggatctctgtctggattcaaggctcaggttcgtctcacacggctattaacaaaagagtctagtttcgtggctgtcttggtcgtgacgctgacgtgagttcacaggagcaattcttcggtcgtgggagtccccgcagcccatccacttcacgccacaaatcattatcggggcggcagtctttggattcgcaggcgcccagcaaaatgaatgccacaggtagttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgggtaccccaagggcgtagatccactagtaacggccgccagtgtgctggaattcgcccttggccgactactagatcgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgtagatcgatcttggtggcgtgaaactcccgcacctcttcggccagcgccttgtagaagcgcgtatggcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcggggccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaaacacggaaggagacaataccggaaggaacccgcgctatccggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcagatctgtggtttgtccattgggttctcggattggcctattacacaaccctggggatctctgtctggattcaaggctcaggttcgtctcacacggctattaacaaaagagtctagtttcgtggctgtcttggtcgtgacgctgacgtgagttcacaggagcaattcttcggtcgtgggagtccccgcagcccatccacttcacgccacaaatcattatcggggcggcagtctttggattcgcaggcgcccagcaaaatgaatgccacaggtagctggcagggctcaagaagtacacgctcccgagcgagggctggttcaagtacttcgtctgcccccactacacattcgaatgcctcctctacttgagccttgccgttgttgcggcgccgacaggatgctgggtgaacggatccatcctgtgctcattgacgtttgtcctggccaatgtcggctcaacggcatacggcaccaagatttggtatgccgaaaagttcggggcggacaaggtggaggggaaatggatcatgattccctttgtattctaataagctactgagcaattcgggaaaacgggttagagcacgctctcatcgcttgccgaagaggcctaatttggctccgactttggacacttggaggacgtctcggtatgtgaggctggtgcgggtctgtctcgggttccgaccgccagcaaacgcctcgggatctcgaagcaagggccagttcacaatggtgtcggcagatagatcaagatcctcctcagcgtcggcgatgccatgcagatagtccgtatagaggttggctgtggtgatgagaagactacgaggttcttggagaatgcgccaggcaggctcgggatcaagggcgccgtcctccttgctgcggtacaggttcaggcagagactcgcgccgaggctgacggtgcacaccacgggccaatacgcagcgccatcctagggtggatgagcatttcgaagctgtcaaggattcttcaactacgtttccataacacttaacttatgcggcatgatgccgattcccggaggatactcgttgataaggacgtgattcgggcgctggtgggggctttcggcgaagatgtgcttgggagagtcaccgtcgtcggagagcggaagggacagtagccgcggcacaatgggggtctcaagccaagagggcagcggtgcgtcgaggagcctgttgttgacgaggtccgacggccaggtctggagccgtcgatgggtgagctgtttccatcgtggacgaggagccgcggcgatctgtgaggaaaaaaaaaagcgtgtgctgtgatcagcagggctgcaggcggagacgaccggcaccatggacggcaccggccacggcggcaagacgcactttgtcaagaatcaggcgctcctcttcctcgctgatgaagtttggcaagtagtaggccgtcgagggcagcgaggcgattcgggcggcttcgagagacgagggcagccacgcgtcgccggtgcctcgagctgccatgattgaaggtatgtaggtaggtatctatgcacggctcgggcaggcaggcaggcaggcaggcagacagacagacagacagacagctgcagtgagttgcaggaggagagctgccttgacagcgccaagagtgagcccaccagtgagcgagaaggcagcctggctcggaatcagatggggggtcgagagggcggacggattgcgagagaagaagcgatcaaatggtcgtaagagggggatgatggggtgatgatccagcgaatggccgtatcgagttaggaggggggctgcagcgcctgtttgctctcgcagcagcacgacttgaacagcgaggtgccggttagggctagccgggcattaatgccttttgtcctaccaaggttgcatggcgcatgtgctgcgcgagctgttcggtcggtggcctcgttatcggcgtgcagcgtctggtcggtccagctgcttgttgctcgaaaggggggaggcgtcgtgtctctgccgtctgcgggaggcggactgggccaggcctgaccgggtcatcgatggatggattgaatggactgaatcatggcgaatgatgatttattgattcattgattgagtgagaaggacggcgaccgatgctgcaggtcgcacagacgaggaaaacagcgcgtggcagcacggcacagcgcggtcggacgcgtgcatgcgtggatatcgagtggtttaaacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 10572 bp, Artificial sequence
  Plasmid pNJOC383
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgtttaaacgcggccgcgaattcatcttgaagttcctattccgagttcctattctctagaaagtataggaacttccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaataagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcagatatccatcacactgggagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagatggtggagagcttataccgagctcccaaatctgtccagatcatggttgaccggtgcctggatcttcctatagaaccatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatccccaagaagaagcgcaaggtctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcacttgcatgcatcctagagtttaaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagac

SEQ ID NO: 13 - DNA, 8029 bp, Artificial sequence
  cbh1 locus with lysozyme cassette inserted between FRT-F and FRT-F3 sites
ggtgaaacaccgcccccttcttgagagagcaacaacaatcattctgctgtcggcagaagagcagagacttgctgaccctagttaatgactacacagctcggaggtttgtgacatgtccatgattttgatacatggcggagagcaatgtggtggacgaaatcaatcaccatatggcgctatattggctgtttcaggtcctgtttcaagctgttcctacagctctttcttggtctacttgtggtcgcctgctacatcagttgatatacccggaattactgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaacggccgcgaattcatcttgaagttcctattccgagttcctattctctagaaagtataggaacttccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaataagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcaactagctagtgcatgcgataacggaatagaagaaagaggaaattaaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacaaacagcataatagcaacagtggaaattagtggcgcaataattgagaacacagtgagaccatagctggcggcctggaaagcactgttggagaccaacttgtccgttgcgaggccaacttgcattgctgtcaagacgatgacaacgtagccgaggaccgtcacaagggacgcaaagttgtcgcggatgaggtctccgtagatggcatagccggcaatccgagagtagcctctcaacaggtggccttttcgaaaccggtaaaccttgttcagacgtcctagccgcagctcaccgtaccagtatcgaggattgacggcagaatagcagtggctctccaggatttgactggacaaaatcttccagtattcccaggtcacagtgtctggcagaagtcccttctcgcgtgcgagtcgaaagtcgctatagtgcgcaatgagagcacagtaggagaataggaacccgcgagcacattgttcaatctccacatgaattggatgactgctgggcagaatgtgctgcctccaaaatcctgcgtccaacagatactctggcaggggcttcagatgaatgcctctgggcccccagataagatgcagctctggattctcggttacgatgatatcgcgagagagcacgagttggtgatggaggggacgaggaggcataggtcggccgcaggcccataaccagtcttgcacagcattgatcttcctcacgaggagctcctgatgcagaaactcctccatgttgctgattgggttgagaatttcatcgctcctggatcgtatggttgctggcaagaccctgcttaaccgtgccgtgtcatggtcatctctggtggcttcgtcgctggcctgtctttgcaattcgacagcaaatggtggagatctctctatcgtgacagtcatggtagcgatagctaggtgtcgttgcacgcacataggccgaaatgcgaagtggaaagaatttcccggcgcggaatgaagtctcgtcattttgtactcgtactcgacacctccaccgaagtgttaagaatggatccacgatgccaaaaagcttgttcatttcggctagcccgtgatcctggcgcttctagggctgaaactgtgttgttaatgtattattggctgtgtaactgacttgaatggggaatgaggagcgcgatggattcgcttgcatgtcccctggccaagacgagccgctttggcggtttgtgattcgaaggtgtgtcagcggaggcgccagggcaacacgcactgagccagccaacatgcattgctgccgacatgaatagacacgcgccgagcagacataggagacgtgttgact

SEQ ID NO: 14 - DNA, 7950 bp, Artificial sequence
  cbh2 locus with lysozyme cassette inserted between FRT-F and FRT-F3 sites
acacttactcttctacacagtcatttcctggagactaacagagctttatgtagtatatatggagacgtgaagctactgccgggtgcatggcttgcccatcaccgcacgagttcgagcacgttaatattccaattacgactcaaatcaatacctttgtcaatgggagctcgtcttgacattaacgcatcctttcaagtaatgcaatgcagcaatggaggaacttgtagagaccgagggaggaatggcgaagggcggccggagcttggagtcctggtggaggctgaaagcttcgagtttcagcgtctcccagaagttacccaacccaagtggctacaacgacaataagtattctatacctagtaatattgttcgatgcttgtatggagtagatgctggagtctggtgtaatattaatggcttagttcatactacatttgacatttccagcccgagagcgcaccgaagccacatgccgcatattgacaaagtgctagattgtgtaaggagggcattctctatagaggaatcagcgtttgcatatacctactacgtcattgccctaatggacagtaagctagccagctgcattatgataagagtaacgtgagataggtaataagtcttacaacactttcccttatagccactaaactacaacatcgtcctgcagttcctatatgatacgtataacccattgatacatccaagtatccagaggtgtatggaaatatcagatcaagacctctctcttctaagaaacctagaaccagacgctggtagtataataagcacactgtgactcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactcggccgcgaattcatcttgaagttcctattccgagttcctattctctagaaagtataggaacttccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaataagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcaactagctagtgcatgccacaatgtcgagtgtctattagacatactccgagaataaagtcaactgtgtctgtgatctaaagatcgattcggcagtcgagtagcgtataacaactccgagtaccagcaaaagcacgtcgtgacaggagcagggctttgccaactgcgcaaccttgcttgaatgaggatacacggggtgcaacatggctgtactgatccatcgcaaccaaaatttctgtttatagatcaagctggtagattccaattactccacctcttgcgcttctccatgacatgtaagtgcacgtggaaaccatacccaaattgcctacagctgcggagcatgagcctatggcgatcagtctggtcatgttaaccagcctgtgctctgacgttaatgcagaatagaaagccgcggttgcaatgcaaatgatgatgcctttgcagaaatggcttgctcgctgactgataccagtaacaactttgcttggccgtctagcgctgttgattgtattcatcacaacctcgtctccctcctttgggttgagctctttggatggctttccaaacgttaatagcgcgtttttctccacaaagtattcgtatggacgcgcttttgcgtgtattgcgtgagctaccagcagcccaattggcgaagtcttgagccgcatcgcatagaataattgattgcgcatttgatgcgatttttgagcggctgtttcaggcgacatttcgcccgcccttatttgctccattatatcatcgacggcatgtccaatagcccggtgatagtcttgtcgaatatggctgtcgtggataacccatcggcagcagatgataatgattccgcagcacaagctcgtatgtgggtagcagaagaactgagcgagatcttcgagggcgtaactctgcatatccgattggcctgctgccacatgtcatttgcttcggtttcttttctgttgagttcttgtatttgggtgaaagtaacatggtgtatgacgagagacattggtggtaagaaaaaatttcacctcctcttagtgcaggactgactctcaaaatctatatgcaaatgtgtcgtgtaacacccttcgcatgagcgctgaccgtaccctaccatttcgccccactcatgatagcagaagagacatattaattcggcaatgctacgaaagtctgcaggtatgcttaaataaacgcttgccacagaagccgacagtttattgttactacttactatactgtattattgttgctcacataaggcggtgaaccattggttcaccacgacgcctgacgaggtaaattactctctcgtagggctgccaaggtaggtcccaaccccgtatcctcggtcgagggtgcgaggttctttggtccttccctctttggtaaagcccagtagcgtgtttgaatcagttcacaatctctcctaaacacagtccgacactaggtaggtacgttgtaatagcaactcaaacatgtaattcgttcaaggcaggaacattttataaac

SEQ ID NO: 15 - DNA, 7936 bp, Artificial sequence
  eg1 locus with lysozyme cassette inserted between FRT-F and FRT-F3 sites
ggagcagatggcgacaccataggcggtgcgaatcgtccagaaacttgttgtgcgagacaccacggtctcatttcgcagaatcccagaccctttatggtcttaccgatgaccggttcaggccaaacagaggcaatggggttctttgagtatatttcgatcaagcatctcaatgaataccgccctagtgattcatggcggagaagcctcatgttcttctcccagacggtaccatccgtgcggtacgctgctatcgcattggcattggccaaccaaaactacctgcatcgcgggtttagcgaccaattgcaccaaccgccatcctcgaaagactggctgtcagacagcgttgccttgttttactataaccgagccatccagcttcttctcaagcaggagaacggagacagcgaggatacaacagccatcacgctcttggtctgttatctcttcatctgctttgatcatctggcgggcaactacgcacaggcaatgaagcacctacgcggaggtgttgagctctcacgaaacgcccaaaaggcgatactagacggcaacaatgcatacgacgagaccaagacctcaggaactcacgcactcatctgccagatcacgagacagatccgtcgtcttgacatgcaagccgtaacgtttctggttgactggactcccgccgatatcaaagacacagccgtatcccagcttgcgcatcccaatagtgcatttcaatctcttgaccaagccgctgaccacctacagagtctcatcgctcgagtcatgagtctacgcaataacccgggacagcaaatgcttcctttgtcagtcaagaatgcagttctcgaacagttggaaacgtggtcgactctcttcgaaaacatgctgcaacaacatggcagttcctctgattcagaggagacggcatacccgctcatcacactgctacgcctacagtataccatcgtatggacttatctcagtagctcagggcctgggagggaaatggaatacgacaacttcctgacccagtttcagcaatgcgtggcgttggcaggcgacttcgcggcagtgcatgagcgatattcggggtcgttgaagccgacgttcacgccggagattggcatgatcccagtgctttacatcatcggggccaaatgccggcatcctgttgtgcggcgggaggccttgggtcttttgaggcggcaaccgatccgggaggcggtttgggatagcgttgttgttgccagggtagtggagaggataatggagattgaggaggttgggtttgagaagtgggaaatgatacagagtatggaacaggttccggtgtggcagagggttgagacgctgtcttgggcacatgtcgtcgtcgatggacagtctgcgggcagagtggacattaactatacgttctgcgcgcgagagggatcgcatattgagtctttcatgatgtaataagcttgggcttgacagcgttctattgccagtgtatcaacgaagtggtatggcggccgcttgaagttcctattccgagttcctattctctagaaagtataggaacttccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaataagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcattaattaagataatggccactttcatctgaatcaaggacaggagccgttgatgatttccagatatcctatcttcatcgctagtactaataacaagtaagcaaacagccagctacactcgtacacaccgctcatgaaaacataagacaaagctcaagccatgtcaagcaccaccccatcgtacacgtacatcttctaccaatctgtccagatgccggctacgtcagcctccaacccattgcgatacaatgacggatgtcgtatagaccatggcaacggctcccaccaccattgtcactgcgtccaaagtcttctcccacctgctctcagcaacgcccttgaaatgcaggaatgccggatacatgtagaccaatggaatgcaggcaaaagatcccgtcagcgccacaaacttatccaaatcacttgctcccaccaccgccacgccgatgcatgccacaatggtcaaggcccggaggccgttcttcttccacttgattgccgcgctcttcttaccagtggcgcgctctccgaaaatcgacgtttccaatatgcggatcgcggggaacagctgcacaggctcgccagccagtacggccagcgagtagagaaactggactgcattgacgagaggcgagtcctggggaaaattggagatgatttgaatcttggtgtgttctccaaacgtcgcatagcagagtgcgccgactgatgtgaagatggccgtgatgaggagcatgacaaagtagaggaggcccttgaactgatgaggcttcttcatactcgactgaattggcaggatcaggccaatgccctcaaacgtgaagatcgccgagcccagagtcaggggaaaatcggaggggttgaagagcttgacgcttggctccataccgtgcctgaccaggcttcgaatgtcaaaataccagatgtatacgagtccaatgaggatgaaggcgtccgccaccagagcaaccggacccagcttggagatgttgcggatccaggctaacggaatgagagggaggaactgaagcgcaattaggcccgacaccccgaaatgaccaaggccgttggtgacagcgtcgaggaaggagaagagattttcagcagtgaagataatgcctgagcagacgaagcccagctgagaaatggcaatggaggcgagaatcaagcttcgaaaccggggtccaacgacggcctcgccaatgtctccatagccaccgccgtacttgtggcggcagtccagaagtaagcggaaacagaagcaattgaccagtgacacggagatgagcgccagtgacgaaaagaggatacctccgttgcggaatgccttgggcaggaacaggatgcctgtgccgatgaaagccttgagcagcgtgaagaaggtcttgactgtgctcgcgtcaccctggcgggcaagacggcgggagcttctccggcgaccagcactcggtcgcgcaagcagcggacgccgctcgtcggcggcttcttccgcctcatcagtgacggcagac

SEQ ID NO: 16 - DNA, 8996 bp, Artificial sequence
  xyn2 locus with lysozyme cassette inserted between FRT-F and FRT-F3 sites
cttgaacgcgaaaatgttatcagggccgaggagatgtaccctgagccttaccagtggccagccatccttcaagggtccaggaagcattggcaaatctggcgagattgaggcatctgctggtataccttgtagttaagggaactcttgtgccttctttgtcaggaacctgccttgcaaggctaagcatctaccttgcttgttcgccttgccttacttagattctgggagcttgtagttacatagctcatatgctatttgcttttgcgaggagggcacttgcaagactggctctatgaagggcgttggatactgtcgctactacggtcgtacgggatggtccttgcaaagaagtgcaatctatttcagaggattgttgttgccttttgagtcttaggttgttgataggacaccttctttacaataaggtagtgaaaaccagcctcttgtcgctgagacttccagttatctggcctacctaggtaggtacccagagaatatctacctaggcagtatggtgctttaaccgtggcccttctgaacctgatctttcttgtcgctctgatttctgctgctcataggctggccttacacttggatctttatggtcaaatgttcatgaaacaacgctctgggtccttgagctatgtatcatgatatcagccttgaacagtctgggttatggctgtaatgcaagcataccatggcttcaaggggcaggagtaaagaaaagaaagacagattctcagagatgatacagactcccttcaaaaacgctgctgtacatgatccccagtgtcgacatcctgatctattctaggtaaggtagattcaactcagtatatcgttattcgagataaaatggatagcattgagtatctgaagaaagtaggtaccaatattaaacttctctttatagctagatcacagtactgaaaagaaaagaagtagcgattattatcttgaataaaagatatagcagtctatatctctactagaggaaagtagaagaagtagagctacctagaattacaagtctcctctttctaccccctcctctcccccctctcctctgcttatttcataaccatctcctgtttcgccatttaaacgtacattcaaagctctctcggcgccgtctgcccacacatcaacaagcatcacgcctgcagcagcatctctaggactgctcgcgcctgaaaagcagatcaacaagagcagttcagactggcattctatccggccctattctcattccgacggttactctgccaccgaccgtgcccagaaggaataccagaacacctgaacctgctggaaggcaccttttagtccaccaactttggaacgacgcagcctcttctctcaaagctcagatcatagacgcagccagcgacatgcagtgcaatgtcatggatgttttgtgggttacgttcgtccctacagagtcactggcaggctacatgcatgtcatggtcccatatcgcccattcgaccaccgtcagcccagtacttgctcgtgtcaatctccgtcagtcgcgtgtgatcagacattccaactggatctgggccagccgggcactctttgccttgtcctgtttgacaggttcaatttgctctgtatgccacttctgtccagtctgtttagtgagccagactgctgagacactctgaaccgaggagcaagcgcctccacagccaagaagttgaagaagacggcagacacccacacagtgaccacgaagctttgtcacgcagtattgatcccatcaaccactagcaaatccctagacaccccgaaaggacttcaacagtgacctagcagcaaagccgtgagtctcggacggcctcttgttcaactcaaccgaccttccaagctacaaaaacgacaaggttggatgtctgccgtttgctgcctcgccaccagctgactttaggcaaaacaggtcattgaatccagatcggagtcgacactcgcatccgtgcggccgcgaattcatcttgaagttcctattccgagttcctattctctagaaagtataggaacttccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaataagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcaactagctagtgcatgcggttgaagggcttgtgttctttggctctgtagaggctctagggggttggattgggctgactatggtctcccctttgtatgctacacactgatcagacgatcgtagtcagttgagagatgagcttcattggtcgatatgagtgctttgagactggacacttgcgtgactgcatgttccgtttcttgtgataattgccgtgaagatgtgtctgctggtatgacgacaaagtagcatcagcagcaaagtatggcgaggactactaggcccaagatattgtcggcttcgtcaaagtgagaaattttagggtctccattgattacattcatccgtttcagaagcacgatatagaggagtagcaccatcaggtcggaaaggtcgtcaacagcttgaacaagtcatactaaggtatgttagtgtggtcgtggttgtcggtatcagtaggtatcattggagtatgtgttatatacagattaatgggaccaatgcatcacgcatcaggacatgccctggcaatcctccctcttacaaatcgcgatgaggcttgggtttgggggtaatagtagcggtcgtaaaggtggtggtgccagtagcgcacacgcagccctggccggaaatgctggtaatgggaggagatttgcaggtgggctcggtggtaaaggaggcgtaggtgctggtgggctcaaccgtgggctcaaccagctcagccttgaccaggacggggatgatctggcctgtggacaggctgaaggagcggtggttgagcacggtgagaatggcgccaatctcgcccgagtggctggtgatgctaatccaggtgttgtcgtcgtgcgaaaagacgtcgtcaatcgcctccttggaacgggcgacctggtgagcaggcgtctcgccctcgtcctctttgccgcgccacagcaggtcctcctcggtgaagccagcctcgaactcaaagttgggagcgtaggcgtgaatctgagacttggtggagcggcggtcgcaggtgtggatgctgatgctctcgcgaaacagctccttgatgacgggtttgaaggggtgtgagcgaggtagcttctggttggcaaaggtgaattgggccgtctggaggcagcgcttgagaggcgagctgtagtacgactcaaaaaaggggaggccttcctgctcgaagcgcgacttgaagtaggcgttggccttggtagtctgctgaatgccgttgggggtcagttcagggtcagcccaggtggaagtgccgttgccatcttgctcggcccagtagcactatggaagcgtaagtcagcagctgtttgagcaacgaagaggagatttgtcgaggctcacattccaggcgggagtgccgtaaaagctttcggcagtgttgtgccatccctcgccgtggcggcccatgaagagcaccttgtaggtgctcttgtcctccttgcggcactcgctgttgagcgactcgacgtagtgggcgaagcgctgccactgggtcttcctgtggtgaggatcgaacctggcatctgtggggtaggtcctgttgatgagaccaaagttgaaggcggcctgtgtcaagcaaagcgtgagtcggagtctctaaccagagcggcacagatgctggcaacacctacatagtcgaagctgccggggtcagtgctcgggtcgtcctggaggaagaagcccggaaccgacgagtacttgatgtcgtcgcagtcggagacgtgcttccaggccacggcgccgcgagcgagcgccagggcgacggacagcagagaggccttcatcttggcgataatgaggatgtagatgaagacgaagaggaggcagaggctcagactcgttggctgcgacggaagacaggcgtcctagagagcaacgtcatgtcgatcccggggcgacgcagcgacttatatgtctgaacctgtccagctgctctctcctgcatgcgacgttgagacactcgcaacaggctggtgaacaccaggatgtaaccagtcgacagggctgggaggctgcacccgctgcgccagatggagcctgtcagtgctgctctggggcctctgtcagcacttg

SEQ ID NO: 17 - DNA, 61 bp, Artificial sequence
  Primer oNJ587
gtaaaacgacggccagtgaattcgagctcgtttaaacgtgctggacctgctaaggaggaag

SEQ ID NO: 18 - DNA, 55 bp, Artificial sequence
  Primer oNJ605
ctgggctgcccgtgaagccgtttaaatgaactacctgtggcattcattttgctgg

SEQ ID NO: 19 - DNA, 53 bp, Artificial sequence
  Primer oNJ606
gcactgtttcttccttgaactctcagatctgtggtttgtccattgggttctcg

SEQ ID NO: 20 - DNA, 50 bp, Artificial sequence
  Primer oNJ607
gcgtgtacttcttgagccctgccagctacctgtggcattcattttgctgg

SEQ ID NO: 21 - DNA, 29 bp, Artificial sequence
  Primer oNJ608
ggtagctggcagggctcaagaagtacacg

SEQ ID NO: 22 - DNA, 63 bp, Artificial sequence
  Primer oNJ609
ggaaacagctatgaccatgattacgccaagcttgtttaaaccactcgatatccacgcatgcac

SEQ ID NO: 23 - DNA, 31 bp, Artificial sequence
  Primer oNJ610
aggtagttcatttaaacggcttcacgggcag

SEQ ID NO: 24 - DNA, 50 bp, Artificial sequence
  Primer oNJ611
gaacccaatggacaaaccacagatctgagagttcaaggaagaaacagtgc

SEQ ID NO: 25 - DNA, 963 bp, Fusarium venenatum
  CDS (1)..(960)
atggaagaattgatacaccaatccatcgatctgatgtcgattttgacaccagcacaatggtgtcagactttcttcgccctttctacggccatcgtccttggtatccaagccctcccacaagatgttcgcagcgctctcatggactacggcgcccgaagacccaaagatgcgaaacacggaaaggagcaagaagaaaacggacagaaagctctcgtgcctctgaggtcatttatgaagaatttaacagagtacgggcaagtgccccattcatggttcttgcacttttacattgtttcggtggccctgtcgggtttctgggcgtggcagtatctcactcaaggtcatgtactgaaaagcattgtgacgtggcagaatcgagctgatggaccgtccatgagcctggagcaaattttcgtggcatggttgctcatggcgttgcagggttcgagaaggctttacgagagtttgtttgtgttcaagccagggtcatcacctatgtggtttattcattgggcacttggtctttcgtactacattgcaatgagccttgctgtctgggtcgagggttccagcgccattttagcagcctgggattctccctatcaacctctccgggttccacgacggctaccttcagccctagcgttatatttcgtggcttacttcaagcaaaaccaatgtcacagacacttggcaagcctcaagaagtataccctcccaagtgagggatggttcaagtatataatctgtccacactatactttagaatgccttgtgtaccttgccattgcatggatcgctgcaccacacggccaaatcttcaacaaaagtatcttgggcgctgtcatgtttgtggctgtgaatctcggtgccacggcaaagggcaccaaggtatggtatgagcagaaattcggtgcaaacaaggtcgctgggcggtggctcatgatcccgcctgtctactag

SEQ ID NO: 26 - protein, 320 aa, Fusarium venenatum
MEELIHQSIDLMSILTPAQWCQTFFALSTAIVLGIQALPQDVRSALMDYGARRPKDAKHGKEQEENGQKALVPLRSFMKNLTEYGQVPHSWFLHFYIVSVALSGFWAWQYLTQGHVLKSIVTWQNRADGPSMSLEQIFVAWLLMALQGSRRLYESLFVFKPGSSPMWFIHWALGLSYYIAMSLAVWVEGSSAILAAWDSPYQPLRVPRRLPSALALYFVAYFKQNQCHRHLASLKKYTLPSEGWFKYIICPHYTLECLVYLAIAWIAAPHGQIFNKSILGAVMFVAVNLGATAKGTKVWYEQKFGANKVAGRWLMIPPVY

SEQ ID NO: 27 - DNA, 10371 bp, Artificial sequence
  Plasmid pNJOC569
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcgtttaaacgtgctggacctgctaaggaggaagttttttttgggacggaaaagcctggtgaggcatgaagatggagagagttttcggtgtgtgaggatttcgagtatcatggaaaaagaaggaattcaagtttgtaacagaaagatgtttaatggttgatttgcgaacatgatagacgtccaaatttcataatcttttgcaattgagccttggagatattacgaaaatgagatgtgagatctcgaaatgttgaggaatattcatgcctggttacacctgccgtcgttgtcatgccactcgggcgggcaggagcatccatgcttcgcctagcaccaaggattggcaaccgccagatggatcatgacggcaggattccttgaatcggttctagacctttttgtgcacgtgccttagctataagccaacgacttgcaaagcggaaggcagcacctgggcatggctgggaaagaagcttgaagcatgcgccttgctgttgccttcgcttggttgatgttatcggggcaacttgcttgagacattggtcgcacttgccgacctgtgaataggcaagacatatttcgtcagtagtacctacccagcgtaagggatttcacgaggaagaaggtgagtcatcgggttcaaatgctgtactacctcttgatggagttgcagatactatcacgagcttgtgtgctactattctgttggaattcggtctcttataccgtccactcggttcagcactgtacgaacaacgtcttcatgctgcaacactggtccatacatcctttgcatccgtcggtcaatcctgcatcgcctcataccaagtccctcctcgtggaaccaagccctctcatcctccttgtctcccagctcttctcaagccatcaaatcccaactgcaacatggaacttgaagttgctgtgaagcaagcgccaatacgacgaatccaccgcccaatcaaaaccccacagcagtgcaacgccacctcagcgtcaattagactacggattggccgtcaagccgtgtatcgtattgctggttgccaagagcctcagtcacaagttcaaaaagcaagcaaatacgcattctctctccttccagtggcaatccatgatttcattgaagccatgttctggtagtaggatctatgcgagctgcagagaagtttctccgcactccatatctgctgcaatggccggcaatcaagtaaatgcaccgagagtcgttggttcaagggacggagcaatgtgccgattttgggaccacaagagcggcggctcagcatgcaggcgaccaaatttcgcacaattgtgcttctccttggtaggggcatatcactccacgcctggctttggcagtgcaattcggcagctttcaaccagcgcaacgaatgtctgccctttcatcccttcctgagtcattttctcccattcactttatctggcccctcaatctctaaggtcttgttcttgtggcctggctgaaccttgcctcgccgcatcacgccagccgcttgaattcacaacttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgggtaccccaagggcgtagatccactagtaacggccgccagtgtgctggaattcgcccttggccgactactagatcgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgtagatcgatcttggtggcgtgaaactcccgcacctcttcggccagcgccttgtagaagcgcgtatggcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcggggccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaaacacggaaggagacaataccggaaggaacccgcgctatccggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcagatctcaagggacggagcaatgtgccgattttgggaccacaagagcggcggctcagcatgcaggcgaccaaatttcgcacaattgtgcttctccttggtaggggcatatcactccacgcctggctttggcagtgcaattcggcagctttcaaccagcgcaacgaatgtctgccctttcatcccttcctgagtcattttctcccattcactttatctggcccctcaatctctaaggtcttgttcttgtggcctggctgaaccttgcctcgccgcatcacgccagccgcttgaattcacaactaagctactgagcaattcgggaaaacgggttagagcacgctctcatcgcttgccgaagaggcctaatttggctccgactttggacacttggaggacgtctcggtatgtgaggctggtgcgggtctgtctcgggttccgaccgccagcaaacgcctcgggatctcgaagcaagggccagttcacaatggtgtcggcagatagatcaagatcctcctcagcgtcggcgatgccatgcagatagtccgtatagaggttggctgtggtgatgagaagactacgaggttcttggagaatgcgccaggcaggctcgggatcaagggcgccgtcctccttgctgcggtacaggttcaggcagagactcgcgccgaggctgacggtgcacaccacgggccaatacgcagcgccatcctagggtggatgagcatttcgaagctgtcaaggattcttcaactacgtttccataacacttaacttatgcggcatgatgccgattcccggaggatactcgttgataaggacgtgattcgggcgctggtgggggctttcggcgaagatgtgcttgggagagtcaccgtcgtcggagagcggaagggacagtagccgcggcacaatgggggtctcaagccaagagggcagcggtgcgtcgaggagcctgttgttgacgaggtccgacggccaggtctggagccgtcgatgggtgagctgtttccatcgtggacgaggagccgcggcgatctgtgaggaaaaaaaaaagcgtgtgctgtgatcagcagggctgcaggcggagacgaccggcaccatggacggcaccggccacggcggcaagacgcactttgtcaagaatcaggcgctcctcttcctcgctgatgaagtttggcaagtagtaggccgtcgagggcagcgaggcgattcgggcggcttcgagagacgagggcagccacgcgtcgccggtgcctcgagctgccatgattgaaggtatgtaggtaggtatctatgcacggctcgggcaggcaggcaggcaggcaggcagacagacagacagacagacagctgcagtgagttgcaggaggagagctgccttgacagcgccaagagtgagcccaccagtgagcgagaaggcagcctggctcggaatcagatggggggtcgagagggcggacggattgcgagagaagaagcgatcaaatggtcgtaagagggggatgatggggtgatgatccagcgaatggccgtatcgagttaggaggggggctgcagcgcctgtttgctctcgcagcagcacgacttgaacagcgaggtgccggttagggctagccgggcattaatgccttttgtcctaccaaggttgcatggcgcatgtgctgcgcgagctgttcggtcggtggcctcgttatcggcgtgcagcgtctggtcggtccagctgcttgttgctcgaaaggggggaggcgtcgtgtctctgccgtctgcgggaggcggactgggccaggcctgaccgggtcatcgatggatggattgaatggactggtttaaacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 52 bp, Artificial sequence
  Primer oNJ588
ctgggctgcccgtgaagccgtttaaatgaagttgtgaattcaagcggctggc

SEQ ID NO: 29 - DNA, 31 bp, Artificial sequence
  Primer oNJ595
cacaacttcatttaaacggcttcacgggcag

SEQ ID NO: 30 - DNA, 60 bp, Artificial sequence
  Primer oNJ596
cccaaaatcggcacattgctccgtcccttgagatctgagagttcaaggaagaaacagtgc

SEQ ID NO: 31 - DNA, 50 bp, Artificial sequence
  Primer oNJ592
gcactgtttcttccttgaactctcagatctcaagggacggagcaatgtgc

SEQ ID NO: 32 - DNA, 50 bp, Artificial sequence
  Primer oNJ593
cccgttttcccgaattgctcagtagcttagttgtgaattcaagcggctgg

SEQ ID NO: 33 - DNA, 34 bp, Artificial sequence
  Primer oNJ589
cacaactaagctactgagcaattcgggaaaacgg

SEQ ID NO: 34 - DNA, 67 bp, Artificial sequence
  Primer oNJ590
ggaaacagctatgaccatgattacgccaagcttgtttaaaccagtccattcaatccatccatcgatg

SEQ ID NO: 35 - DNA, 21 bp, Artificial sequence
  Primer oNJ632
cagccaagaagtgctcggctc

SEQ ID NO: 36 - DNA, 25 bp, Artificial sequence
  Primer AgJg685
ctacatcgaagctgaaagcacgaga

SEQ ID NO: 37 - DNA, 20 bp, Artificial sequence
  Primer oNJ633
gatgtggtgctgcagccagc

SEQ ID NO: 38 - DNA, 23 bp, Artificial sequence
  Primer AgJg604
gggacgccctgctgcaacttacc

SEQ ID NO: 39 - DNA, 10679 bp, Artificial sequence
  pTmmD-Tl_lipase Thermomyces lanuginosus HL703 lipase variant expression cassette flanked by FRT-F and FRT-F3 sites
gcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgtttaaacgcggccgcgaattcatcttgaagttcctattccgagttcctattctctagaaagtataggaacttcgctagccgagagcagccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcacatgaagttcttcaccaccatcctcagcaccgccagccttgttgctgctctccccgccgctgttgactcgaaccataccccggccgctcctgaacttgttgcccggagtcctattcgtcgagaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagaatactggatcaaatctggaacccttgtccccgtccggcgacgagacatcgtgaagatagaaggcatcgatgccaccggcggcaataaccagcctaacattccggatatccctgcgcacctatggtacttcgggttaattgggacatgtctttagtaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcagatatccatcacactgggagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagatggtggagagcttataccgagctcccaaatctgtccagatcatggttgaccggtgcctggatcttcctatagaaccatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatccccaagaagaagcgcaaggtctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcacttgcatgcatcctagagtttaaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 40 - DNA, 12973 bp, Artificial sequence
  Plasmid pSMai326 containing an Paenibacillus sp. xanthanase variant expression cassette flanked by FRT-F and FRT-F3 sites
gcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgtttaaacgcggccgcgaattcatcttgaagttcctattccgagttcctattctctagaaagtataggaacttcgctagccgagagcagccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgaagcttcttccctccttgattggcctggccagtctggcgtccctcgccgtcgccatcgccggcgtcgtccagagcgtcaacgtcagccaggccggctacagcagcaacgacttcaagaccgccaccgtcaccgccagcgacaagctcagcgacaccagctaccagatcctccagggcaccaccgtcattgccaccggcaccatgaaggacgagggctacgtctggggcaagtacgtctacagcatcgacttctccagcgtcacggccacgggcaccaacttcaccatccgcagcaacggcgtcagcagctacacgttccccatccagaccaacatgtggaacgagtacaaggacgagatgaccgccttctaccgcctcctccgcaccaccgacaccttcgccgcctatcctgccggctactccaacattgcccctagcaacaagattctgcaccccgactcgttcctcgacgacgccttctcgcccgaccgcaccacgcactacgacctcaccggcggctggttcgatgccggcgactacggcaagtatggcggcaaccagtgggtccagggcaacattgctatcagctacctccgccacgccagctctgccgccgtcaacttcgacaaggacaccaacggcatccccgacctggtcgacgaggccatcttcggcagccagtacctcgtcaagttcgcccagcagcttggcggcgctatccacaacatcctccgcaagggcggcttcgtcctgcctcacaaggtcaccgacaacgtccccggcaacaccgacgatcgcgccctcgaggctgtcgaggccgtcggcggcagcggcaagagcagcggcagcctcgccgccaccgctcgcgccatccgcaccgctattgctggcggcaaggtcgccgccaacaaggtggcccagctgcagaccctcgccaacgagttccaggccgctgccatcatcttctacaactacaccctcacgcaccaggacggcaaccacggcagctacggcacgatgaacaacggcggcattgctaacccgctcctctgggccgaggtccagctctacctcctgaccggcgacgccgcctacaagacgcaggcccagacgcgcatcaacgccatcaacgaggcctacgtgagcagcaccaactactgggacatgcaccccattgctctcgccgagttctaccccgtcgccgacagcgccatcaagaccaagatccagagcatcctgaagcaccaggcctactacttcatcaccctcatggacgacacgccctacggcgtcctcaaccagttcggcaacttcggcgtcaacgagccccacgcctcctacatggccgacctgctccgctactacgagctgttcaacgaccctgtcgctctccgagccgccaagaaggccctctactggatcgtcggcaacaacccctggaacatcagctgggtcagcggcgtcggcagcaactttacccgcttcctgcacacccgcctcgacgaggaggcctacagccagacgaacacgggcgtcgtgctccctggcgccatggtgagcggccccaacatcaaggaccccaacaacaagctgagcagcagcccctggtacgaggacaagcccatttgggccgacgacacgaaccagtggcgctacaacgagtactccgtcagcatccagacgggcctcttctacaccatcatgggcctcagcgccctcggcggcaacgcctccactggcggcgctgagcccgtcaagctccccatcacgtggcccatcatcggcgactgggtcacgggcgacgtcaccgtctttgcccagcctgagggcagcctctcgaacgtcagcgccaacggcaccgtgctcagccccagcgacggcgtctacaccaccacggtcagcaccagcgccgacgctccctacaccgagcgcaaggtccagatcaagggcaccgacgactcgggcaacaccacctacagcaacacccacttcacggtcgcccctccgctgcctgatccttcgcaccctctcctcttcgacgactttaaccagaagggcatctggggcagccagaagctcgattgggtcaactggtacaaccagaacggcggcaccgccagctacacccgcaccacggtcgacggccgcaccgtcggcaagttcgctcacacccctgccgccaccaccagcaaggccaagtttcagccccgcaagtacaacgccaacctcaacggctaccgctacctcaacttcacgatgaagaaccctggctaccccaacacgaagatccgcattgccgccaacgacggcaccaagtccgtcaacctcacctccggcgaggtcgccatcagcagcacctggaccacctaccagtacgacctgaacctccatcctacgctcaacaagagcaacgtcctcatcgaggtctggctcagcaaccccacggctggcgcctacggcgagatcctgatcgacgagatctctgccgtcaacaccaacagcggcacggcccctacgctctccgccaccggcgtgaacgccagcattggcaaccagagcaccgtgttcacctacaccgccacgtacaccgacgccaacaaccaggctcctttcgacgtccaggtcgtcattgacggcgtcatccgctccatgaccgctgctgaccccacggacacgacctactccgacggcaaggtttacacctacgccacgaagctccccgtcggcacccacaagttctacttccgcacgacggacaccacgaccaacttcgtcagcacctccgtccagactggccccacggtcatccgcaaccgactcgaggccgaggtgctcagcatcaacctgacgaacggcacccatgccgtcaaggacaacgccgacgccagcggcggcaagtaccgcctcttcaacggccgccaggccaacgactacattgagtacgccgtgaacgtccccaaggccggcacgtaccaggtcagcgcccgagccatgcgcctctccgataacggcatctatcagctccagattaacggctccaaccagggcacgcccttcgacacttaccagtcctcgggcaagtatctcgactacgccctgggcaacgtcaccatcacgagccccggcacgcagctgttccgcttcaaggtgaccggcaagaacgcctcgagcctcggctacaagctgcccctggactttatccagctcgtccccgtgccttaaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcagatatccatcacactgggagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagatggtggagagcttataccgagctcccaaatctgtccagatcatggttgaccggtgcctggatcttcctatagaaccatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatccccaagaagaagcgcaaggtctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcacttgcatgcatcctagagtttaaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 41 - DNA, 10427 bp, Artificial sequence
  Plasmid pTmmD-Mf_Lysozyme containing an Myceliophthora fergusii lysozyme expression cassette flanked by FRT-F and FRT-F3 sites
gcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgtttaaacgcggccgcgaattcatcttgaagttcctattccgagttcctattctctagaaagtataggaacttcgctagccgagagcagccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcacatgaaggctgccctcctcgctacggtgtcggctcttgctgctggcgttcaagccgctgtccagggtttcgacatcagccactggcagtcttctgtcgacttcaaggctgcgtacaactctggcgctcgcttcgtgatcatcaaggctaccgaaggaacctcgttcatcgatcccaagttctcgagccattacactggtgctaccaatgctggcttcattcgtggtgcctatcatttcgcccatcccggtcaaagctctggagaagcgcaagccgactacttcctcgcgcatggcggtggctggactccggacggcatcacccttcctggcatgttggacctggaggcctacaatgcgggtgaatgctggggtctcagccagagcgcgatggtggcctggatcaaggccttctccgatcgctatcatgcgcgtacgggagtctacccgatgctgtacaccaacctctcgtggtggaagacgtgcacgggcaactctaaggccttcgtgaacaccaatcccttggtcctggctcgatgggcttccagccctggcgagattcctggcggatggccatggcagaccatttggcagaactccgacagctatcgctatggcggtgactcggacatcttcaacggcgacatgaaccagcttcgccgactcgctacggctgcttaataagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcatcaagcttggtaccgagctctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagacaagggcgaattctgcattgaagttcctattccgagttcctattcttcaaatagtataggaacttcagatatccatcacactgggagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagatggtggagagcttataccgagctcccaaatctgtccagatcatggttgaccggtgcctggatcttcctatagaaccatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatccccaagaagaagcgcaaggtctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcacttgcatgcatcctagagtttaaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 42 - DNA, 41 bp, Artificial sequence
  Primer IH1232-3′steD-F
aactctctcctctagattatgtagcatgagaccagcgggga

SEQ ID NO: 43 - DNA, 45 bp, Artificial sequence
  Primer IH1233-3′steD-R
acaggagaattcttaattaaagtccggggtggggagttttcaggc

SEQ ID NO: 44 - DNA, 44 bp, Artificial sequence
  Primer IH1230-5′steD-F
gtggcggccgcgtttaaacatccctattttaaataccgagtatg

SEQ ID NO: 45 - DNA, 44 bp, Artificial sequence
  Primer IH1231-5′steD-R
tcagtcacccggatccctaatggtggcagtcgtgttggatgcct

SEQ ID NO: 46 - DNA, 11166 bp, Artificial sequence
  Plasmid pIhar473
ttaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgcgtttaaacatccctattttaaataccgagtatgctggggtagtacacacgcatacacaacaacacaccggacggaggagaggaaaactgaagttcctccaagtcctcctccgccttctcccttcttcccttcccccctccggacctccgaagtcccggttatttgggagggattgtttattgcaggagatgagaggccgctttagtctcgggtctagctcgcaagctgcttccttctcacgagaccctggtggatcaaacacacgccgattcctactcggatggggagcgcctgtttatgatttatgactgctcatctcgctcatccccttggctgtggagtggtttcggtggtttttctgttctccacatctcgagctttttgaacgtgtcgcatcatcatgacagcctataccctttgtaaccgtaatattaccagagcctcctccggtgccatgataccgtccggcccggggcgcagcttgctctctttgcctctctctcgcgcatcttctccagcttctctcctgttgtctagactcttgtcaccaaatttaggagccggcggaactggtgaccattagctgttctcttctcccccccctctcttctccctccgctgcaaaagtcctactccgcattggcggcggactccggtcgcaatggagctccccttcagcctggtggatacagctctttcctccacgcacattgacgccatcgacgcccttcgagcctttttccttttggcatcctgcacggtatgcaccgatatgttgccatcccaaattcgaggcggacgacgttgcaaagtgaccctgcatcaactaaacccagaatatcccaactagatactctccgtcagcatcctgccagattcgcttcattcacgcttcattccttatggcgctcgtacaacttcgacggccgactcgcagtcctcgcagtcctcgcccccggcgtcctcggactcctccccagccgcccgcgctcttgactacgctgccgccttgacagtccctcacagctacttcacgcaattctacattgcctccgttttggcctcggtcttctgggctgcgcagcttctgtgtcaaggagccgtgttccaggcctttgcaacgagaatcagtccggaacatatgcaacagtccatgtccatacaccaagtcttcttgtgctgggcgctcatgctcattcagggtgcaaggcggctatatgagtgcaaggccttcgccaagccttcagcctcaagaatgtggtttgttcactggctcgtaggccttgcattttacctagcagtatccgttgccttatggatcgaaggagcaggtaccactctgactgacaggctaaaataccgaaccccactgattgcatctaggagccctgctgtctcataaagtgattctcgacgacctcaaggttacaatagcaccctctctccgcacctttgtctgcattcctcttttcttgattgcatcaggcatccaacacgactgccaccattagggatccgggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcctctagtactaaatgacgtttgtgaacagcccaaagcctacaaattcaactgcgcacaacgcgcccacggcaacttcctcgagaacgcgccgcagacaatgctctctatcctggtggcaggcgtcaagtacccagaggcagcagcgggcttaggagcggcctgggttgttctccgcaccctctacatgctgggctatatttatagcgacaagccgaacggcaccggcaggtacaatggttcgctgtacttgcttgcgcaagcgggtctttggggattgagcgcatttggtgttgcaaaggatttgatgtaaatgtagtcgacatcttagcacagaggggagagttgataaaatgtggtctgtttgaatgatagtcgggttcgtgacctatattcgtgatagtggagataggtctgcgcctatcttatcgggccggagcaaaaattccaccgcagcggggtgagttttcgttatacagccatcccacttccagcttcaaattgtcagtttaatccagcccaattcaatcattggagaaccgccatcatgtcttcgaagtcccacctcccctacgcaattcgcgcaaccaaccatcccaaccctttaacatctaaactcttctccatcgccgaggagaagaaaaccaacgtcaccgtctccgcagacgttactacttccgccgagctcctcgatcttgctgaccgcctaggcccctatatcgcagttctgaaaacccacatcgacatcctcaccgatctcaccccgtcgaccctttcctcgctccaatccctcgcgacaaagcacaacttcctcatctttgaggaccgcaagttcatcgacatcggcaacaccgtgcaaaagcagtaccacggtggcgctctccgcatctccgaatgggcacacatcatcaactgcgccatcctgccgggcgaagggatcgtcgaggccctcgcacagacaaccaagtctcctgactttaaagacgcgaatcaacgaggtctcctgattcttgccgagatgacgagtaagggatctcttgcgacaggggagtacacggcacgctcggttgagtacgcgcggaagtataaggggtttgtgatgggattcgtgagtacaagggcgttgagtgaggtgctgcccgaacagaaagaggagagcgaggattttgtcgtctttacgactggggtgaatctgtcggataagggggataagctggggcagcagtatcagacacctgggtcggcggttgggcgaggtgcggactttatcattgcgggtaggggcatctataaggcggacgatccagtcgaggcggttcagaggtaccgggaggaaggctggaaagcttacgagaaaagagttggactttgagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcctctagattatgtagcatgagaccagcggggaaattggcctgagacatgagccccggatgcgtcgcgtcaccgactgccgaggaacttcatcgtattcccgagactggcaactttgagcacatctcgataagtcaagcttatcctagtctccctctccagccagccgcactcatatctgtctggctcccgcaacaaatcccagttgcagatggattctgcgccaagcccttcatccctggtggtctctgcaattccatgcagaaagtcggtatagatactcttcgtggtaacgagcaggctgcgcctttcctgcaaaatcctatacaccggacgccggccatgtccgctccctttgccatcctcgcttctctcgtacagatctagcacgataggagctcccaggctcacagtagctacgagcgggtagtaggcagcaccgtcttcatgcggcattatgccctgtcccgggcggtattcatttaccagaacatgattcggggcgtggtgcggggcgtccgcgaaaagacccaaggagtcgaagcgaggggtgatgatgggtgagacgagccaagagggcaacggggaggagatgagcgcgttcgattttgtcagggccgaaggccaggtttgaagtcgccggtgagctagatgagtccagcggggtaacggcgcagtcgttatctatagccgggggtccatgagcgcccaacatccccacagggcctgaatgaacttaggccactgcacagtaaagccacggtaactcactcacctttcggaggagaaactcctcttcatcctccgagatgaaatccgctatgtagaatccatcgtccggcagtgatttgatgcgggcggtctcaagatcggctctcatttgttggagtcaatcccggggagcacacggagcaaatatccctgttgttcaatggggggtcggatgacgactgggaggagattaatctatcagaacgagtccgttgagttggttacacggtaagccgggcgatcagacagggggggtgtggtcggtcacggtgaaagaaggggatcgtagtgaaacttaccgggcgttgagcgtagacccggaacccagacgggatgaatggtaagggaagtgatgatgccgatgaatggcatgtcgtcacgatgagacgcaatggctgcccacttggccgagctgtggtacgtaatccgtagcgttaactccatcacaatggattgatgcccagtgactgttcaattagtcgtgtgggtgataagaatagaagtcactttcccctcacctgacatctgcggacagctcatgactcgacaaaaagcttatctatcaactatactccggatcaaagttacttcaatcatcaaaataaacaatgcctgagcatcacataaacatacacaaatatatatatatacccatatggatatagatgaacccctcgttctaccatgatgcaaacagtatgttcaacccagtagcctgaaaactccccaccccggactttaattaagaattctcctgtaggcttgagagttcaaggaagaaacagtgcaattatctttgcgaacccaggggctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagactctgctaagctattcttctgcttcgccggagcctgaagggcgtactagggttgcgaggtccaatgcattaatgcattgcagatgagctgtatctggaagaggtaaacccgaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcgatccggatagcgcgggttccttccggtattgtctccttccgtgtttcagttagcctcccccatctcccgggcaaacgtgcgcgccaggtcgcatatcgtcggtatggagccgggggtggtgacgtgggtctggaccatcccggaggtaagttgcagcagggcgtcccggcagccggcgggcgattggtcgtaatccaggataaagacgtgcatggaacggaggcgtttggccaagacgtccaaggcccaggcaaacacgttatacaggtcgccgttgggggccagcaactcggggccccgaaacagggtaaataacgtgtccccgatatggggtcgtgggcccgcgttgctctggggctcggcaccctggggcggcacggccgtccccgaaagctgtccccagtcctcccgccacgacccgccgcactgcagataccgcaccgtattggcaagtagcccgtaaacgcggcgaatcgcagccagcatagccaggtccagccgctcgccggggcgctggcgtttggccaggcggtcgatgtgtctgtcctccggaagggccccaagcacgatgttggtgccgggcaaggtcggcgggatgagggccacgaacgccagcacggcctggggggtcatgctgcccataaggtaccgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccgggcggacttccgtggcttcttgctgccggcgagggcgcaacgccgtacgtcggttgctatggccgcgagaacgcgcagcctggtcgaacgcagacgcgtgttgatggccggggtacgaagccatacgcgcttctacaaggcgctggccgaagaggtgcgggagtttcacgccaccaagatcgatctacagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggccgttattactcagcccttctctctgcgtccgtccgtctctccgcatgccagaaagagtcaccggtcactgtacagagctcaagcttcgattaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattca

SEQ ID NO: 47 - DNA, 25 bp, Artificial sequence
  Primer IH1252-ste-proF
atactctccgtcagcatcctgccag

SEQ ID NO: 48 - DNA, 25 bp, Artificial sequence
  Primer IH1253-ste-500R
ctgctccttcgatccataaggcaac

SEQ ID NO: 49 - DNA, 20 bp, Artificial sequence
  Primer HTJP-324
aagggatgcaagaccaaacc

SEQ ID NO: 50 - DNA, 22 bp, Artificial sequence
  Primer HTJP-325
tgaagaatttgtgttgtctgag

SEQ ID NO: 51 - DNA, 83 bp, Artificial sequence
  Primer oAT3303
tgctgcggaacaaggggtgggagggaagggtgtacttcttgagagagttaagctagtggtgggcgtcatgttgtaggccagat

SEQ ID NO: 52 - DNA, 17132 bp, Artificial sequence
  Plasmid pAT3631
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcgctacataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtacccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaagagatttcgactagttatgtacctggagtttggatataggaatgtgttgtggtaacgaaatgtaagggggaggaaagaaaaagtcgtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtaccaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgacgactttttctttcctcccccttacatttcgttaccacaacacattcctatatccaaactccaggtacataactagtcgaaatctctttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccgggaagatctcatggtcatagctgtttccgttaattaatggttcacttctctttagaaatcaactgtgggttttgctttttgcttcattctctttgtcttctccatctttgatcaaatcctggactttctcaatccccagctaattcaatcatagtcagttttctatttttattatttctttttcttttgaaatgtgattaacaaccagtccgttatatatcttgtacccagattacgcccaactcgtgctcctcagccacaaagatactcaattgatagccaagatacatacataccacaaagtaaggactccatgcattgagtattactcatcgtattctagactactccaaaactcagcacatagacaaacaatacgaacctcgtctaggggtgattcagaggcggcaaagcggggttttcgcatttgatgttcctggcacttatgtaagcccacgcttcccgctcaactaaaccatcagccaatcagactgctcagatttatcttttgaagggtaaataaatcattgtaaagaagaacaagtggcttgcttgtcaagcaatggcatcattggtctagtggtagaattcgtcgttgccatcgacgaggcccgtgttcgattcacggatgatgcaggaatttctactcttgtagattctctcaagaagtacaccctttttttttttgagcatttatcagcttgatatagaggtaggaatgtatggaggtgcagaatggctattttgttattggagcgggttcgaaacggagggcaggagactttttctaaatacgtcacgtgatatagagctgctttaattaagacctcagccgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagagtattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgtccatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcacattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatccaatgcgcgattcaagacattaaaaatggaaaatttcaaaaaattgcgcccgcagcgcgtgcatacaaaattcatcccaatacaggggaccagacaggcgccactcggccgggccacagctgcttgggtgttgaccgggagcggaccaattaaggactcgaacgaccgcggggttcaaatgcaaacaagtacaacacgcagcaaacgaagcagcccaccactgcgttgatgcccagtttgactgtccgaaatccaccggaaaggtggaaacatactatgtaacaatcagagggaagaaaaaatttttatcgacgaggcaggacagtgactgatggtggggtcatggtcgggtctccgagcgaaagagaaccaaggaaacaagatcaacgaggttggtgtacccaaaaggccgcagcaacaagagtcatcgcccaaaagtcaacagtctggaagagactccgccgtgcagattctgcgtcggtcccgcacatatcgggaagcaagacttgagcgccgagagaagcagttagactatcgaacacgaatgctggatgtgttagatgagagattggatgatgccttgcgccgacgcatgtccctgagagacgctggtccgtattactctcgacagtattatgaaaattattgatttgagttctcttttccttgcatttttcggtttttatgattccccttctgtttgaatgttcctcttatcctcgggccgctttttctgttagtttctattattcttttcgggtgtgagtgggggatagacactaataacgtcattcaccggggaccgttttgtataccagtgttgtcgcgacctgttgcgtttagtgtatgttccacgtatgtactttctctaaaaaaaagtcgaatgagtcatgagaatgagatatgatatgcaccttaccgcactataactgggatcattgtatagtatggatctgtagaggacaggatgttcagggccaagtcagcagttgacagcgcattgcatccggtgacgagaacttatcgataagccccaccagtgccggcctcaggcagtccaacccccgccatagagtgggattgatttggttcgtttttcttccgtcttccaccttttatgtcatcgctcttccctgtcgtctgatcttcttctactctttcttccatactgaacttgacaatcaccctgtcttttctcaatcaaactcgtcgtatcttacttccaccggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacgcgttctaggatccgtcgacctgcagccaagctttcgcgagctcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcgctagcacaattgaggcatccccactaccgcattaagacctcagcgcggccgcaaatttaaataaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 53 - DNA, 21 bp, Artificial sequence
  A. oryzae steroid dehydrogenase genomic protospacer
tctctcaagaagtacaccctt

SEQ ID NO: 54 - DNA, 17 bp, Artificial sequence
  Primer oAT3163
ctagcagtctcaatcgc

SEQ ID NO: 55 - DNA, 17 bp, Artificial sequence
  Primer oAT3164
ttgaccgtgacaaagac

(19) United States
(12) Patent Application Publication
(10) Pub. No.: US 2022/0025423 A1
(43) Pub. Date: Jan. 27, 2022

(54) Modified Filamentous Fungal Host Cells

(71) Applicant: Novozymes A/S, Bagsvaerd (DK)

(72) Inventors: Nicholas Jochumsen, Lyngby (DK); Michael Rey, Davis, CA (US); Chiho Inoue, Ichikawa-shi City (JP)

(73) Assignee: Novozymes A/S, Bagsvaerd (DK)

(21) Appl. No.: 17/296,938

(22) PCT Filed: Nov. 26, 2019
(86) PCT No.: PCT/US2019/063415

§ 371 Date: May 25, 2021

Related U.S. Application Data
(60) Provisional application No. 62/772,301, filed on Nov. 28, 2018.

(51) Int. Cl. C12P 21/02; C12N 9/04; C12N 15/80

(52) U.S. Cl. CPC C12P 21/02; C12N 9/0006; C12R 2001/685; C12Y 101/01; C12N 15/80

(57) ABSTRACT

The present invention relates to mutated filamentous fungal host cell producing a secreted polypeptide of interest, wherein a native putative steroid dehydrogenase is modified, truncated, partly or fully inactivated, present at reduced level or eliminated compared to a non-mutated parent cell, and wherein said native putative steroid dehydrogenase comprises at least one conserved amino acid motif selected from: YGAR and/or VPHS[W/Y]F and/or QC[A/V/S]RRL and/or LKKYTLP and/or CPHYT, and methods of producing a secreted polypeptide of interest in said cells as well as methods of producing said cells.

### REFERENCE TO SEQUENCE LISTING

This application contains a Sequence Listing in computer readable form. The computer readable form is incorporated herein by reference.

### FIELD OF THE INVENTION

The present invention relates to modified filamentous fungal cells and to methods for producing such cells as well as methods of producing secreted polypeptides of interest therein.

### BACKGROUND OF THE INVENTION

Filamentous fungal host cells are widely employed for the industrial production of a wide variety of polypeptides of interest. Intense research efforts are directed at improving the production of polypeptides of interest in filamentous fungal host cells, especially into improving productivity and/or yield.

### SUMMARY OF THE INVENTION

The instant inventors found that modifying a gene encoding a predicted putative steroid dehydrogenase in an enzyme-producing host cell led to improved productivity and/or yield of the enzyme.

Accordingly, in a first aspect, the present invention is directed to mutated filamentous fungal host cells producing a secreted polypeptide of interest, wherein a native putative steroid dehydrogenase is modified, truncated, partly or fully inactivated, present at reduced level or eliminated compared to a non-mutated parent cell, and wherein said native putative steroid dehydrogenase comprises at least one conserved amino acid motif selected from: YGAR and/or VPHS[W/Y]F and/or QC[A/V/S]RRL and/or LKKYTLP and/or CPHYT; preferably said native putative steroid dehydrogenase comprises at least two of the conserved motifs; more preferably at least three or four of the conserved motifs; most preferably said native putative steroid dehydrogenase comprises all five of the conserved motifs.

Modifying, truncating, inactivating, reducing the level of or completely eliminating the native putative steroid dehydrogenase may be done by any suitable method known in the art, such as, reducing expression of the encoding gene by replacing the native promoter of said gene with a heterologous promoter, preferably a regulated promoter. Another strategy to reduce the level of the putative steroid dehydrogenase could be to add destabilization domains such as ubiquitin domains to the protein and thereby reduce the half-life of the protein. Yet another way to inactivate, reduce the level of or completely eliminate the native putative steroid dehydrogenase is to co-express or add one or more steroid dehydrogenase inhibitor. Examples of convenient ways to completely eliminate expression are gene deletion, gene replacement or gene interruption, e.g. by introducing a non-sense mutation in the coding sequence. Another way to modify the coding sequence could be to introduce an internal deletion, either by deleting some of the coding sequence, or by tampering with intron processing by mutating the coding sequence. Yet another way to inactivate the putative steroid dehydrogenase could be to silence its expression using RNA interference or siRNA or by insertion of a construct containing a promoter and possibly lacking a terminator with direction of transcription towards the end of the gene encoding the putative steroid dehydrogenase resulting in sterical hinderance of transcription of the gene encoding the putative steroid dehydrogenase due to colliding RNA polymerases or possible mRNA destabilization due to formation of mRNA molecules with complementary sequences.

Accordingly, in a second aspect, the invention relates to methods of producing a mutated filamentous fungal host cell having an improved yield of a secreted polypeptide of interest compared with a non-mutated parent host cell, said method comprising the following steps in no particular order:
a) transforming a filamentous fungal host cell with a polynucleotide construct encoding the secreted polypeptide of interest; and
b) mutating the host cell to modify, truncate, partly or fully inactivate, reduce the level of or eliminate a native putative steroid dehydrogenase, wherein said native putative steroid dehydrogenase comprises at least one conserved amino acid motif selected from: YGAR and/or VPHS[W/Y]F and/or QC[A/V/S]RRL and/or LKKYTLP and/or CPHYT; preferably at least two of the conserved motifs; more preferably at least three or four of the conserved motifs; most preferably said native putative steroid dehydrogenase comprises all five of the conserved motifs.

A final aspect of the invention relates to methods of producing a secreted polypeptide of interest, said method comprising the steps of:
a) cultivating a mutated filamentous fungal host cell according to any preceding claim under conditions conducive to the production of the secreted polypeptide; and, optionally,
b) recovering the secreted polypeptide of interest.

### BRIEF DESCRIPTION OF THE FIGURES

FIG. 1 shows a plasmid map of pNJOC577.

FIG. 2 shows a plasmid map of pNJOC383.

FIG. 3 shows a multiple alignment of the four putative steroid dehydrogenases identified in SEQ ID NOs:3, 6, 9 and 12. Identical residues are indicated by black boxes. Residues conserved in three out of four proteins are indicated by gray boxes. The proteins are aligned using the MUSCLE algorithm version 3.8.31 with default parameters (Edgar, R.C. (2004). Nucleic Acids Research, 32(5), 1792-1797).

FIG. 4 shows a plasmid map of pNJ00569.

FIG. 5 shows the relative lysozyme productivity/yield (LSU(F)/ml) for strains NJOC587 (control) and NJOC618-81D (steroid dehydrogenase mutant). The LSU(F)/ml data for the control at the end of fermentation was used to normalized the data. In the figure, commas have been used as decimal separators instead of the traditional decimal point.

FIG. 6 shows a plasmid map of pTmmD-TI_lipase.

FIG. 7 shows the relative lipase productivity/yield (LU(LXP)/ml) for strains NJOC600-2A (control) and NJOC609-1A (steroid dehydrogenase mutant). The LU(LXP)/ml data for the control at the end of fermentation was used to normalized the data. In the figure, commas have been used as decimal separators instead of the traditional decimal point.

FIG. 8 shows a plasmid map of pSMai326.

FIG. 9 shows the relative xanthanase productivity/yield for strains NJOC608-1B (control) and NJOC617-77C (steroid dehydrogenase mutant). The xanthanase yield for the control at the end of fermentation was used to normalized the data. In the figure, commas have been used as decimal separators instead of the traditional decimal point.

FIG. 10 shows a plasmid map of pTmmD-M.f. Lysozyme.

FIG. 11 shows the relative M.f. lysozyme productivity/yield (LSU(A)/ml) for strains NJOC601-5A (control) and NJOC610-2B (steroid dehydrogenase mutant). The lysozyme yield for the control at the end of fermentation was used to normalized the data. In the figure, commas have been used as decimal separators instead of the traditional decimal point.

FIG. 12 shows a plasmid map of pIHAR473.

FIG. 13 shows a plasmid map of pAT3631.

FIG. 14 shows the relative phytase productivity/yield for strains AT3091 (control) and AT3944 (steroid dehydrogenase mutant). The phytase yield for the control at the end of fermentation was used to normalized the data. In the figure, commas have been used as decimal separators instead of the traditional decimal point.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

### Host Cells

The present invention relates to recombinant host cells comprising a polynucleotide of the present invention operably linked to one or more control sequences that direct the production and secretion of a heterologous polypeptide of interest.

A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell of the invention is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus aculeatus, Aspergillus aculetinus, Aspergillus awamori, Aspergillus brasiliensis, Aspergillus foetidus, Aspergillus fumigatus, Aspergillus japonicus, Aspergillus luchuensis, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

In one aspect, the invention relates to mutated filamentous fungal host cell producing a secreted polypeptide of interest, wherein a native putative steroid dehydrogenase is modified, truncated, partly or fully inactivated, present at reduced level or eliminated compared to a non-mutated parent cell, and wherein said native putative steroid dehydrogenase comprises at least one conserved amino acid motif selected from: YGAR and/or VPHS[W/Y]F and/or QC[A/V/S]RRL and/or LKKYTLP and/or CPHYT; preferably said native putative steroid dehydrogenase comprises at least two of the conserved motifs; more preferably at least three or four of the conserved motifs; most preferably said native putative steroid dehydrogenase comprises all five of the conserved motifs.

In a preferred embodiment of the aspects of the invention, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocaffimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma; even more preferably the filamentous fungal host cell is an Aspergillus aculeatus, Aspergillus aculetinus, Aspergillus awamori, Aspergillus brasiliensis, Aspergillus foetidus, Aspergillus fumigatus, Aspergillus japonicus, Aspergillus luchuensis, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Preferably, the secreted polypeptide of interest is native or heterologous; preferably the secreted polypeptide is an enzyme; preferably the enzyme is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, xylanase, or beta-xylosidase.

In a preferred embodiment of the invention, the native putative steroid dehydrogenase comprises or consists of an amino acid sequence at least 60% identical to the mature amino acid sequence shown in SEQ ID NO:3, SEQ ID NO:6, SEQ ID NO:9 and/or SEQ ID NO:12; preferably at least 65%, 70%, 75%, 80%, 85%, 90%, 95%, 96%, 97%, 98% or most preferably at least 99% identical to the mature amino acid sequence shown in SEQ ID NO:3, SEQ ID NO:6, SEQ ID NO:9 and/or SEQ ID NO:12.

Preferably, the native putative steroid dehydrogenase is encoded by a gene comprising or consisting of a nucleotide sequence at least 60% identical to the genomic DNA sequence shown in SEQ ID NO:1, SEQ ID NO:4, SEQ ID NO:7 and/or SEQ ID NO:10; preferably at least 65%, 70%, 75%, 80%, 85%, 90%, 95%, 96%, 97%, 98% or most preferably at least 99% identical to the genomic DNA sequence shown in SEQ ID NO:1, SEQ ID NO:4, SEQ ID NO:7 and/or SEQ ID NO:10.

Alternatively, the native putative steroid dehydrogenase is encoded by a gene comprising or consisting of nucleotide sequence at least 60% identical to the cDNA sequence shown in SEQ ID NO:2, SEQ ID NO:5, SEQ ID NO:8 and/or SEQ ID NO:11; preferably at least 65%, 70%, 75%, 80%, 85%, 90%, 95%, 96%, 97%, 98% or most preferably at least 99% identical to the cDNA sequence shown in SEQ ID NO:2, SEQ ID NO:5, SEQ ID NO:8 and/or SEQ ID NO:11.

In a preferred embodiment, the native putative steroid dehydrogenase has been modified, truncated, partly or fully inactivated, present at reduced levels compared to a non-mutated parent cell or eliminated by non-sense or frameshift mutation of the encoding gene, by partial or complete deletion of the encoding gene or by silencing of the encoding gene.

In a second aspect, the invention relates to method of producing a mutated filamentous fungal host cell having an improved yield of a secreted polypeptide of interest compared with a non-mutated parent host cell, said method comprising the following steps in no particular order:
a) transforming a filamentous fungal host cell with a polynucleotide construct encoding the secreted polypeptide of interest; and
b) mutating the host cell to modify, truncate, partly or fully inactivate, reduce the level of or eliminate a native putative steroid dehydrogenase, wherein said native putative steroid dehydrogenase comprises at least one conserved amino acid motif selected from: YGAR and/or VPHS[W/Y]F and/or QC[A/V/S]RRL and/or LKKYTLP and/or CPHYT; preferably at least two of the conserved motifs; more preferably at least three or four of the conserved motifs; most preferably said native putative steroid dehydrogenase comprises all five of the conserved motifs.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase (amdS), Aspergillus oryzae neutral alpha-amylase (e.g., amyB), Aspergillus oryzae acid stable alpha-amylase (asaA), Aspergillus oryzae or Aspergillus awamori glucoamylase (glaA), Aspergillus oryzae TAKA amylase, Aspergillus oryzae alkaline protease (a/pA), Aspergillus oryzae triose phosphate isomerase (tpiA), Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus oryzae neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus oryzae glucoamylase, Aspergillus oryzae alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus oryzae glucoamylase, Aspergillus oryzae alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus oryzae neutral amylase, Aspergillus oryzae glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (npr7), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus oryzae glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

Another efficient way to ensure site-specific genomic integration is the use of FRT sites, for example, FRT-F and FRT-F3, inserted at each of the genomic loci for site-specific targeted integration of an expression cassette using the Saccharomyces cerevisiae flippase (FLP) and FRT flippase recognition sequences as described in WO 2012/160093 and US 2018/0037897.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Removal or Reduction of Activity

The present invention also relates to methods comprising a step of mutating the host cell to modify, truncate, partly or fully inactivate, reduce the level of or eliminate the putative steroid dehydrogenase, wherein said native putative steroid dehydrogenase comprises at least one conserved amino acid motif selected from: YGAR and/or VPHS[W/Y]F and/or QC[A/V/S]RRL and/or LKKYTLP and/or CPHYT; preferably at least two of the conserved motifs; more preferably at least three or four of the conserved motifs; most preferably said native putative steroid dehydrogenase comprises all five of the conserved motifs.

The mutant cell may be constructed by reducing or eliminating expression of the polynucleotide or a homologue thereof using methods well known in the art, for example, insertions, disruptions, replacements, or deletions. In a preferred aspect, the expression of the polynucleotide is altered, reduced or eliminated. The polynucleotide to be altered, reduced or eliminated may be, for example, be mutated or modified in the coding region or a part thereof essential for activity, or in a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the polynucleotide or homologue thereof may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame or intron processing. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

Methods for deleting or disrupting a targeted gene are described, for example, by Miller, et al (1985. Mol. Cell. Biol. 5:1714-1721); WO 90/00192; May, G. (1992. Applied Molecular Genetics of Filamentous Fungi. J. R. Kinghorn and G. Turner, eds., Blackie Academic and Professional, pp. 1-25); and Turner, G. (1994. Vectors for Genetic Manipulation. S. D. Martinelli and J. R. Kinghorn, eds., Elsevier, pp. 641-665).

An example of a convenient way to eliminate or reduce expression of a polynucleotide is based on techniques of gene replacement, gene deletion, gene editing or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention also relates to methods of inhibiting the expression of a polypeptide having activity in a cell, comprising administering to the cell or expressing in the cell a double-stranded RNA (dsRNA) molecule, wherein the dsRNA comprises a subsequence of an polynucleotide or homologue thereof. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA for inhibiting transcription. In another preferred aspect, the dsRNA is micro RNA for inhibiting translation.

The present invention also relates to such double-stranded RNA (dsRNA) molecules, comprising a portion of the mature polypeptide coding sequence of SEQ ID NO:1 and/or SEQ ID NO:4 and/or SEQ ID NO:7 and/or SEQ ID NO:10 for inhibiting expression of the polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi); see, for example, U.S. Pat. No. 5,190,931.

The dsRNAs of the present invention can be used in gene-silencing. In one aspect, the invention provides methods to selectively degrade RNA using a dsRNAi of the present invention.

The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824 and 6,515,109.

The protease-deficient mutant cells are particularly useful as host cells for expression of heterologous secreted polypeptides.

The methods used for cultivation and purification of the product of interest may be performed by methods known in the art.

### Methods of Production

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

One aspect of the invention relates to methods of producing a secreted polypeptide of interest, said method comprising the steps of:
a) cultivating a mutated filamentous fungal host cell according to any preceding claim under conditions conducive to the production of the secreted polypeptide; and, optionally,
b) recovering the secreted polypeptide of interest.

In a preferred embodiment, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma; even more preferably the filamentous fungal host cell is an Aspergillus cell; preferably an Aspergillus aculeatus, Aspergillus aculetinus, Aspergillus awamori, Aspergillus brasiliensis, Aspergillus foetidus, Aspergillus fumigatus, Aspergillus japonicus, Aspergillus luchuensis, Aspergillus nidulans, Aspergillus niger or Aspergillus oryzae.

Preferably, the secreted polypeptide of interest is an enzyme; preferably the enzyme is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

### EXAMPLES

### Filamentous Fungal Strains

Trichoderma reesei BTR213 has been described in WO 2013/086633.

Trichoderma reesei strain frt4new-1940-1996-2012-12-1 is a ku70 disrupted and paracelsin synthetase (pars) deleted strain of T. reesei BTR213. The cellobiohydrolase I (cbh1), cellobiohydrolase II (cbh2), endoglucanase I (eg1), and xylanase II (xyn2) genes are deleted in this strain and has FRT sites (FRT-F and FRT-F3) inserted at each of these four loci for site-specific targeted integration of an expression cassette using the Saccharomyces cerevisiae flippase (FLP) and flippase recognition sequences FRT-F and FRT-F3 as described in WO 2012/160093 and US 2018/0037897. The endoglucanase II (egg) and endoglucanase III genes (eg3) are deleted in this strain as well. The Aspergillus niger cytosine deaminase (fcyA) gene is inserted between the FRT-F and FRT-F3 sites at each of the four loci to use as counterselection on 5-fluorocytosine (5-FC).

### Media and Solutions

COVE plates were composed of 342.30 g of sucrose, 25 g of Difco™ agar Noble, 20 ml of COVE salts solution, 10 mM acetamide, 15 mM cesium chloride and deionized water to 1 liter. The solution was sterilized by autoclaving.

COVE2 plates were composed of 30 g of sucrose, 20 ml of COVE salts solution, 10 ml of 1 M acetamide, 25 g of Difco™ agar Noble, and deionized water to 1 liter. The solution was sterilized by autoclaving.

COVE2 glucose plates containing 5-fluorocytosine (5-FC) (Sigma Chemical Co.) were composed of 20 ml of COVE salts solution, 10 ml of 1 M acetamide, 25 g of Difco™ agar Noble, and deionized water to 1 liter. The solution was sterilized by autoclaving. Forty ml 50% (w/v) glucose (sterile) was added after autoclaving. The solution was cooled to 50° C. and 5-FC was added to a final concentration of 75 μg/ml.

COVE salts solution was composed of 26 g of KCl, 26 g of MgSO₄.7H₂O, 76 g of KH₂PO₄, 50 ml of COVE trace metals solution, and deionized water to 1 liter. The solution was sterilized by autoclaving.

COVE trace metals solution was composed of 0.04 g of Na₂B₄O₇.10H₂O, 0.4 g of CuSO₄.5H₂O, 1.2 g of FeSO₄.7H₂O, 0.7 g of MnSO₄.H₂O, 0.8 g of Na₂MoO₂.2H₂O, 10 g of ZnSO₄.7H₂O, and deionized water to 1 liter. The solution was sterilized by autoclaving.

Fermentation batch medium was composed of 24 g of dextrose, 40 g of soy meal, 8 g of (NH₄)₂SO₄, 3 g of K₂HPO₄, 8 g of K₂SO₄, 3 g of CaCO₃, 8 g of MgSO₄.7H₂O, 1 g of citric acid, 8.8 ml of 85% phosphoric acid, 1 ml of anti-foam, 14.7 ml of trace metals solution, and deionized water to 1 liter.

PDA plates were composed of 39 g of Difco™ potato dextrose agar and deionized water to 1 liter. The solution was sterilized by autoclaving.

PDA+1 M sucrose plates were composed of 39 g of Difco™ potato dextrose agar, 342.30 g sucrose and deionized water to 1 liter. The solution was sterilized by autoclaving.

PEG buffer was composed of 50% polyethylene glycol (PEG) 4000, 10 mM Tris-HCl pH 7.5, and 10 mM CaCl₂ in deionized water. The solution was filter sterilized.

Sample buffer (pH 7.5) was composed of 0.1 M Tris-HCl, 0.1 M NaCl and 0.01% Triton X-100. The solution was filter sterilized. Shake flask medium was composed of 20 g of glycerol, 10 g of soy meal, 1.5 g of (NH₄)₂SO₄, 2 g of KH₂PO₄, 0.2 g of CaCl₂, 0.4 g of MgSO₄.7H₂O, 0.2 ml of trace metals solution, and deionized water to 1 liter.

1.2 M sorbitol was composed of 218.4 g sorbitol and deionized water to 1 liter. The solution was sterilized by autoclaving.

STC was composed of 1 M sorbitol, 10 mM Tris-HCl pH 7.5, and 50 mM CaCl₂ in deionized water. The solution was filter sterilized.

TBE buffer was composed of 10.8 g of Tris Base, 5 g of boric acid, 4 ml of 0.5 M EDTA pH 8, and deionized water to 1 liter.

TE buffer is composed of 1 M Tris-HCl pH 8.0 and 0.5 M EDTA pH 8.0.

Trace metals solution was composed of 26.1 g of FeSO₄.7H₂O, 5.5 g of ZnSO₄.7H₂O, 6.6 g of MnSO₄.H₂O, 2.6 g of CuSO₄.5H₂O, 2 g of citric acid, and deionized water to 1 liter. The solution was sterilized by autoclaving.

Trichoderma Minimal Media (TrMM) plates with 1.5 μM 5-fluoro-2′-deoxyuridine (FdU) were composed of 20 ml of COVE salts solution, 0.6 g of CaCl₂.2H₂O, 6 g of (NH₄)₂SO₄, 25 g of Difco™ agar Noble, and deionized water to 1 liter. The solution was sterilized by autoclaving. Following autoclaving, 40 ml of sterile 50% (w/v) glucose was added. The media was cooled to 50° C. and FdU (sterile) was added to a final concentration of 1.5 μM.

2×YT+Amp plates were composed of 16 g of Bacto™ tryptone, 10 g of Bacto™ yeast extract, 5 g of NaCl, 15 g of Bacto™ agar, 1 ml of ampicillin at 100 mg/ml (filter sterilized, was added after autoclaving), and deionized water to 1 liter. The solution was sterilized by autoclaving.

YP medium was composed of 1% Bacto™ yeast extract and 2% Bacto™ peptone in deionized water. The solution was sterilized by autoclaving.

YPD medium was composed of 1% Bacto™ yeast extract, 2% Bacto™ peptone and 2 glucose. The solution was sterilized by autoclaving.

Fermentation feed medium was composed of 1190 g glucose, 14.2 ml 85% H3PO4 and 486 g H₂O. The solution was sterilized by autoclaving.

### Example 1: Genomic DNA Extraction from Trichoderma reesei

Trichoderma reesei was grown in 50 ml of YPD medium in a 250 ml baffled shake flask at 28° C. for 2 days with agitation at 200 rpm. Mycelia from the cultivation was collected using a MIRACLOTH® (EMD Chemicals Inc.) lined funnel, squeeze-dried, and then transferred to a pre-chilled mortar and pestle. Each mycelia preparation was ground into a fine powder and kept frozen with liquid nitrogen. A total of 1-2 g of powder was transferred to a 50 ml tube and genomic DNA was extracted from the ground mycelial powder using a DNEASY® Plant Maxi Kit (QIAGEN Inc.). Five ml of Buffer AP1 (QIAGEN Inc.) pre-heated to 65° C. was added to the 50 ml tube followed by 10 μl of RNase A 100 mg/ml stock solution (QIAGEN Inc.) and incubated for 2-3 hours at 65° C. A total of 1.8 ml of AP2 Buffer (QIAGEN Inc.) was added and centrifuged at 3000-5000×g for 5 minutes. The supernatant was decanted into a QIAshredder Maxi Spin Column (QIAGEN Inc.) placed in a 50 ml collection tube, and centrifuged at 3000-5000×g for 5 minutes at room temperature (15-25° C.) in a swing-out rotor. The flow-through in the collection tube was transferred, without disturbing the pellet, into a new 50 ml tube. A 1.5 ml volume of Buffer AP3/E (QIAGEN Inc.) was added to the cleared lysate, and mixed immediately by vortexing. The sample (maximum 15 ml), including any precipitate that may form, was pipetted into a DNEASY® Maxi Spin Column (QIAGEN Inc.) placed in a 50 ml collection tube and centrifuged at 3000-5000×g for 5 minutes at room temperature (15-25° C.) in a swing-out rotor. The flow-through was discarded. Twelve ml of Buffer AW (QIAGEN Inc.) was added to the DNEASY® Maxi Spin Column, and centrifuged for 10 minutes at 3000-5000×g to dry the membrane. The flow-through and collection tube were discarded. The DNEASY® Maxi Spin Column was transferred to a new 50 ml tube. One-half ml of Buffer AE (QIAGEN Inc.), pre-heated to 65° C., was pipetted directly onto the DNEASY® Maxi Spin Column membrane, incubated for 5 minutes at room temperature (15-25° C.), and then centrifuged for 5 minutes at 3000-5000×g to elute the genomic DNA. The concentration and purity of the genomic DNA was determined by measuring the absorbance at 260 nm and 280 nm.

### Example 2: Trichoderma reesei Protoplast Generation and Transformation

Protoplast preparation and transformation of Trichoderma reesei were performed using a protocol similar to Penttila et al., 1987, Gene 61: 155-164. Briefly, T. reesei was cultivated in two shake flasks, each containing 25 ml of YPD medium, at 27° C. for 17 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 30 ml of 1.2 M sorbitol containing 5 mg/ml of Yatalase™ (Takara Bio USA, Inc.) and 0.5 mg/ml of Chitinase (Sigma Chemical Co.) 60-75 minutes at 34° C. with gentle shaking at 75-90 rpm. Protoplasts were collected by centrifugation at 834×g for 6 minutes and washed twice with cold 1.2 M sorbitol. The protoplasts were counted using a hemocytometer and re-suspended to a final concentration of 1×10⁸ protoplasts per ml of STC.

Approximately 1-10 μg of DNA was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. For DNA containing a hygromycin B resistance marker (hph), the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days.

### Example 3: Construction of Plasmid (pNJOC577; SEQ ID NO:13) for Modification of the TrA1331W Gene (SEQ ID NO:1) Encoding the Putative Steroid Dehydrogenase

A plasmid for modification of the protein encoded by TrA1331W (SEQ ID NO:1) by introduction of a mutation leading to a either a truncation or an internal deletion of a number of consecutive amino acids in the region encoding a putative steroid dehydrogenase domain was constructed by cloning a 5′ targeting region, a hph (hygromycin phosphotransferase) and a tk (HSV-1 thymidine kinase) cassette, a repeat to be used for excision of the hph and the tk cassette and a 3′ targeting region into pUC19 (linearized with HindIII and SacI) using an NEBuilder® HiFi DNA Assembly Cloning Kit (New England Biolabs®, Inc.). The 5′ targeting region, the hph and tk cassette, the repeat for excision of the hph and tk cassette and the 3′ targeting region were PCR amplified using the primer sets:

oNJ587 (SEQ ID NO:19)+oNJ605 (SEQ ID NO:20),

oNJ610 (SEQ ID NO:25)+oNJ611 (SEQ ID NO:26),

oNJ606 (SEQ ID NO:21)+oNJ607 (SEQ ID NO:22), and

oNJ608 (SEQ ID NO:23)+oNJ609 (SEQ ID NO:24).

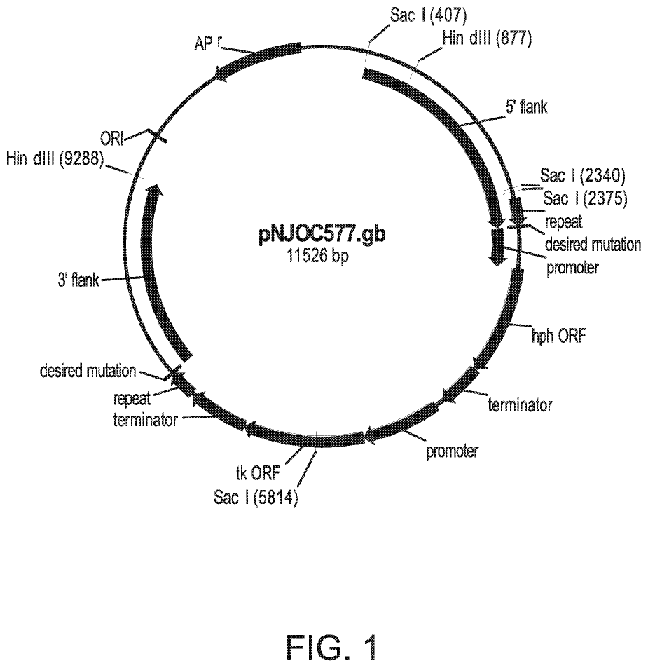

The amplification reactions were performed using Phusion® Hot Start II DNA Polymerase (Thermo Fisher Scientific) according to the manufacturer's instructions. The PCRs were composed of 5 ng of pJfyS1579-41-11 (WO 2010/039840) (template for the hph/tk cassette) or 50 ng of BTR213 genomic DNA (WO 2013/086633) as template, 1×HF buffer, 200 μM of each dNTP, 500 nM forward primer, 500 nM reverse primer, 1 unit of Phusion® Hot Start II DNA Polymerase and sterile Milli-Q® H₂O was added to a final volume of 50 μl. The reactions were incubated in a Bio-Rad C1000 Touch™ Thermal Cycler (Bio-Rad Laboratories) programmed for 1 cycle at 98° C. for 3 minutes; 35 cycles each at 98° C. for 10 seconds, 65° C. for 30 seconds and 72° C. for 30 seconds (the repeat fragment) or 2.5 minutes; and one cycle at 72° C. for 5 minutes. Following thermocycling, the PCR products were separated by 1% agarose gel electrophoresis in TBE buffer and the bands (2354 bp, 4395 bp, 322 bp and 1986 bp) corresponding to the different PCR products were excised from the gel and purified using a NucleoSpin® Gel and PCR Clean-up kit (Macherey-Nagel) according to the manufacturer's instructions. pUC19 was digested with HindIII and SacI in a 50 μl reaction composed of 5 μg pUC19, 20 units each of HindIII-HF (New England Biolabs®, Inc.) and SacI-HF (New England Biolabs®, Inc.), 1× CutSmart® buffer (New England Biolabs®, Inc.) and sterile Milli-Q® H₂O to a final volume of 50 μl. The reaction was incubated at 37° C. and then subjected to 1% agarose gel electrophoresis in TBE buffer. The 2645 bp pUC19 HindIII/SacI fragment was excised from the gel and purified using a NucleoSpin® Gel and PCR Clean-up kit (Macherey-Nagel) according to the manufacturer's instructions. The PCR products and the pUC19 HindIII/SacI fragment were fused together using a NEBuilder® HiFi DNA Assembly Master Mix kit (New England Biolabs) in a total volume of 30 μL composed of 1× NEBuilder® HiFi Assembly Master Mix and 0.04 μmol of each PCR product. The reaction was incubated at 50° C. for 45 minutes and then placed on ice. One μL of the reaction was used to transform 60 μL Stellar™ Competent Cells (Clontech Laboratories, Inc.) according to the manufacturer's instructions. The transformation reaction was spread onto two 2×YT+Amp plates and incubated at 37° C. overnight. Putative transformant colonies were isolated from the selection plates and plasmid DNA was prepared from each one using a QIAprep Spin Miniprep kit (Qiagen) and screened for proper insertion of the fragments by digestion with PvuII. Plasmid DNA giving rise to the expected band pattern (4991 bp, 3362 bp, 2364 bp, 439 bp and 370 bp) upon restriction enzyme digestion was used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to a model of the pNJOC577 plasmid (SEQ ID NO:13) using the Map Reads to Reference module with a high-stringency setting. The Basic Variant Detection module was used to detect the presence of any single nucleotide polymorphism. A plasmid having the expected nucleotide sequence was named pNJOC577 (FIG. 1).

### Example 4: Construction of Trichoderma reesei Strain with Modification of the Protein Encoded by TrA1331W (NJ00586)

Trichoderma reesei frt4new-1940-1996-2012-12-1 protoplasts were generated as described in example 2. Approximately 2-4 μg of linearized TrA1331W modification cassette from pNJOC577 (8871 bp PmeI fragment) was added to 100 μl of protoplast solution and mixed gently. PEG buffer (250 μl) was added, and the reaction was mixed and incubated at 34° C. for 30 minutes. STC (1 ml) was then added and the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days. Next, hygromycin resistant transformants were transferred to PDA plates and incubated at 30° C. for 5-7 days. Transformants were screened for correct integration of the TrA1331 modification cassette by spore PCR. For each transformant, spores were collected with a sterile 1 μl inoculation loop and suspended in 20 μl of Dilution buffer (PHIRE™ Plant Direct PCR Kit, Thermo Scientific) in a thin-walled PCR tube. Each spore suspension was used as template in a PCR reaction to screen for integration of TrA1331W modification cassette at the TrA1331W locus. Two PCRs were performed for each transformant; one for the 5′ site of integration and one for the 3′ site of integration. The 5′ integration screen was performed using a primer annealing to a region upstream the site of integration and a primer annealing to a region within the hph and tk cassette. The 3′ integration screen was performed using a primer annealing to a region downstream the site of integration and a primer annealing to a region within the hph and tk cassette. Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and H₂O to a final volume of 20 μl. Thermocycling was performed according the manufacturer's instructions. The PCR products were analyzed by 1% agarose gel electrophoresis using TBE buffer. Transformants giving rise to the desired PCR products were then subjected to single spore isolation on PDA+1 M sucrose plates. The plates were incubated for 3-5 days at 30° C. Spores from individual colonies were transferred to new PDA plates and the plates were incubated at 30° C. for 5-7 days. The 5′ and 3′ integration verification PCRs described above were repeated and the PCR products were analyzed by 1% agarose gel electrophoresis using TBE buffer. The TrA1331W modification construct pNJOC577 contains the hph and HSV-1 tk cassette flanked by direct repeats to facilitate spontaneous loop out of the hph and HSV-1 tk cassette and generation of a clean TrA1331W modification via homologous recombination between the two repeats. Spores from transformants with correct integration of the TrA1331W modification cassette were collected in H₂O and dilutions were spread onto TrMM plates containing 1.5 μM 5-fluoro-2′-deoxyuridine (FdU) and incubated at 30° C. for 5 days to facilitate identification of isolates having lost the hph and HSV-1 tk cassette. FdU-resistant isolates were then transferred to PDA plates and loss of the hph and HSV-1 tk cassette was verified by spore PCR. In this spore PCR, three primers were added; one primer annealing to a region upstream the hph and tk cassette, one primer annealing to a region outside the 3′ integration site and one primer annealing to a region within the hph and tk cassette. The primers were designed to yield a short or a long PCR product depending upon if the hph and tk cassette is still present. Transformants having lost the hph and tk cassette were then subjected to single spore isolation on PDA+1 M sucrose plates. The plates were incubated for 3-5 days at 30° C. Spores from individual colonies were transferred to new PDA plates and the plates were incubated at 30° C. for 5-7 days. To confirm the presence of the desired mutation in the TrA1331W gene, genomic DNA was prepared for a few isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to a model of the TrA1331W gene (SEQ ID NO:1) using the Map Reads to Reference module with a high-stringency setting. The presence of the desired mutation was verified using the Basic Variant Detection module. One of the isolates containing the desired mutation was named NJ00586 and saved for further studies.

### Example 5: Transformation of Trichoderma reesei Strain frt4new-1940-1996-2012-12-1 with pNJOC383

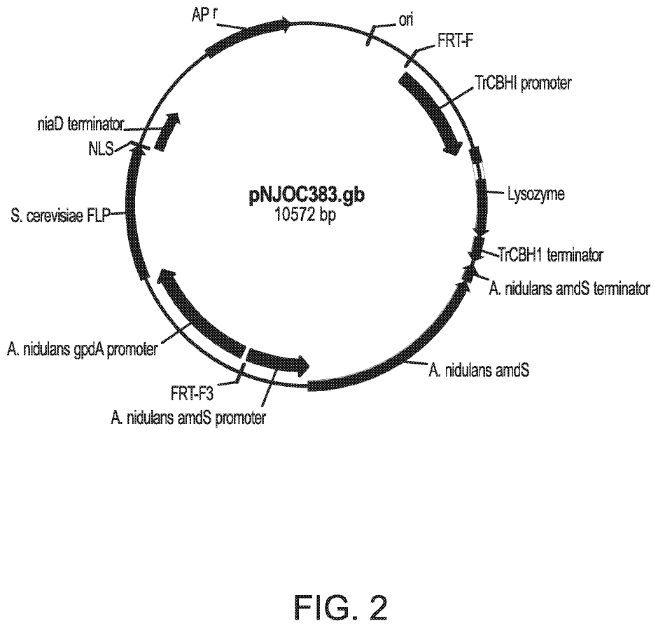

Trichoderma reesei frt4new-1940-1996-2012-12-1 protoplasts were generated as described in example 2. Approximately 1-10 μg of pNJOC383 (plasmid containing an Acremonium alcalophilum CBS114.92 lysozyme expression cassette flanked by FRT-F and FRT-F3 sites for FLP-mediated integration at four loci containing the FRT-F and FRT-F3 sites in the host strain; SEQ ID NO:14 and FIG. 2) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC587 and saved for further studies.

### Example 6: Transformation of Trichoderma reesei TrA1331W Modified Strain (NJ00586) with pNJOC383

Trichoderma reesei NJ00586 protoplasts were generated as described in example 2. Approximately 1-10 μg of pNJOC383 (SEQ ID NO:14; FIG. 2) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NO:15-18) using the Map Reads to Reference module with a high-stringency setting. One of the isolates having correct integration at all four sites was named NJ00588 and saved for further studies.

### Example 7: Lysozyme Activity Assay (LSU(F)/Ml)

Whole broth from fermentation was mixed for roughly 2 hours in a rotisserie mixer at 30° C. After whole broth mixing, all samples were diluted 100× in pre-dilution buffer, then mixed for roughly 2 hours using the rotisserie mixer again. Next, the 100× pre-diluted samples were diluted 10000× in 0.1 M Tris-HCl, 0.1M NaCl, 0.01% Triton X-100 buffer pH 7.5 (sample buffer) by 10-fold serial dilutions followed with a series of 3× dilutions down to 1/9 of the diluted sample. This method was used in conjunction with a Beckman Coulter Biomek FX and SpectraMax plate reader from Molecular Devices. A lysozyme standard was diluted from 0.05 LSU(F)/ml concentration and ending with a 0.002 LSU(F)/ml concentration in the sample buffer. A total of 50 μl of each dilution including standard was transferred to a 96-well flat bottom plate. Fifty micro-liters of a 25 ug/ml fluorescein-conjugated cell walls substrate solution was added to each well then incubated at ambient temperature for 45 minutes. During the incubation, the rate of the reaction was monitored at 485 nm (excitation)/528 nm (emission) for the 96-well plate at 15-minute intervals. Sample concentrations were determined by extrapolation from the generated standard curve.

### Example 8: Lab-Scale Fermentation Showed that Modification of the Protein Encoded by TrA1331W Leads to Increased Lysozyme Productivity/Yield

The four-copy lysozyme expressing strains NJOC587 and NJ00588 were evaluated in 2 liter fermentations. Each strain was grown on two PDA plates for 5-7 days at 30° C. Three 500 ml shake flasks, each containing 100 ml of Shake Flask medium, were inoculated with two plugs per shake flask from a PDA plate. The shake flasks were incubated at 28° C. for 48 hours on an orbital shaker at 200 rpm. The cultures were used as seed for fermentation.

A total of 150 ml of each seed culture was used to inoculate 3-liter glass jacketed fermentors (Applikon Biotechnology) containing 1.5 liters of Fermentation Batch medium. The fermentors were maintained at a temperature of 28° C. and pH was controlled using an Applikon 1030 control system to a set-point of 3.5+/−0.1. Air was added to the vessel at a rate of 2.5 L/min and the broth was agitated by Rushton impeller rotating at 300-1100 rpm. Fermentation feed medium composed of autoclaved glucose and phosphoric acid was dosed at a rate of 0 to 14 g/L/hour for a period of approximately 7 days. Aliquots of whole broth were taken on days 5, 6 and 7 and stored at 5 to 10° C. until they were processed for lysozyme activity assay.

The lysozyme expression level was determined as described in Example 7. Increased lysozyme expression was observed in the NJ00588 strain compared to the NJOC587, which showed that the described modification of the TrA1331W gene encoding the native putative steroid dehydrogenase is beneficial for lysozyme expression.

### Example 9: Identification of Homologs of the TrA1331W Encoded Protein (SEQ ID NO:3)

The amino acid sequence of the protein (SEQ ID NO:3) encoded by TrA1331W was used to perform BLAST searches (E-value: 1.00e−5 and wordsize: 5) for homologs of the protein encoded by genes in the genomes of Aspergillus niger, Aspergillus oryzae and Fusarium venenatum. A single BLAST hit was obtained for each organism and is presented in table 1:

TABLE 1
Homologues of the protein encoded by TrA1331W identified
in A. niger, A. oryzae and F. venenatum.

| SEQ ID NO: | Organism |
|---|---|
| 6 | A. niger |
| 9 | A. oryzae |
| 12 | F. venenatum |

The percent identity between the different proteins was calculated using the Needleman-Wunsch algorithm as described above. The identities between the proteins are summarized in table 2.

TABLE 2
Amino acid %-identity matrix. Identities are presented as percent identity calculated
using the Needleman-Wunsch algorithm as described elsewhere herein.

| | T. reesei | A. niger | A. oryzae | F. venenatum |
|---|---|---|---|---|
| | [SEQ ID | [SEQ ID | [SEQ ID | [SEQ ID |
| | NO: 3] | NO: 6] | NO: 9] | NO: 12] |
| T. reesei [SEQ ID NO: 3] | 100 | 43.00 | 39.23 | 54.78 |
| A. niger [SEQ ID NO: 6] | 43.00 | 100 | 70.19 | 44.52 |
| A. oryzae [SEQ ID NO: 9] | 39.23 | 70.19 | 100 | 42.19 |
| F. venenatum [SEQ ID 12] | 54.78 | 44.52 | 42.19 | 100 |

According to table 2, the proteins share significant sequence identity, ranging from approximately 39% to 70% identity (the highest percent identity was not surprisingly observed between the more closely related A. niger and A. oryzae proteins).

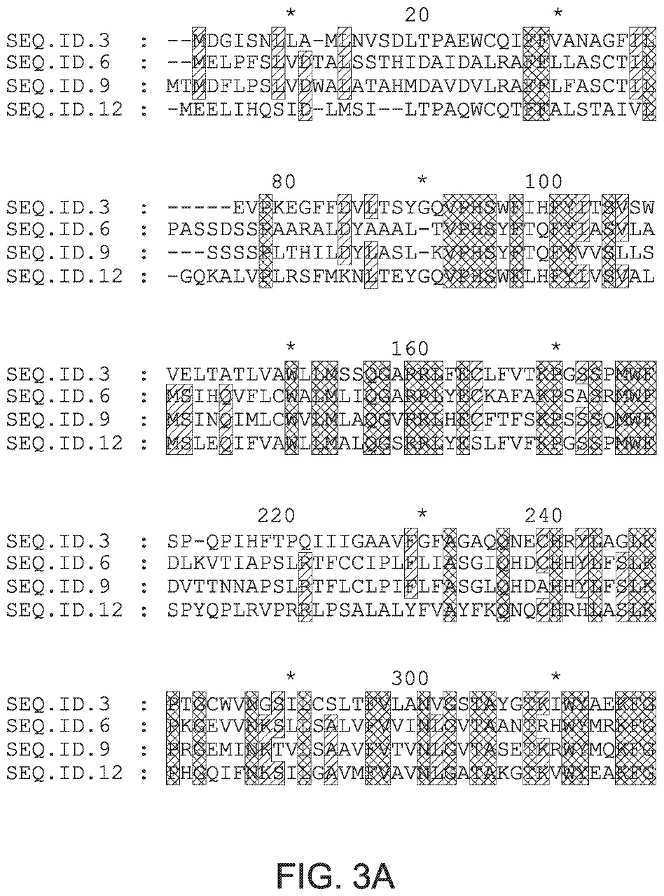

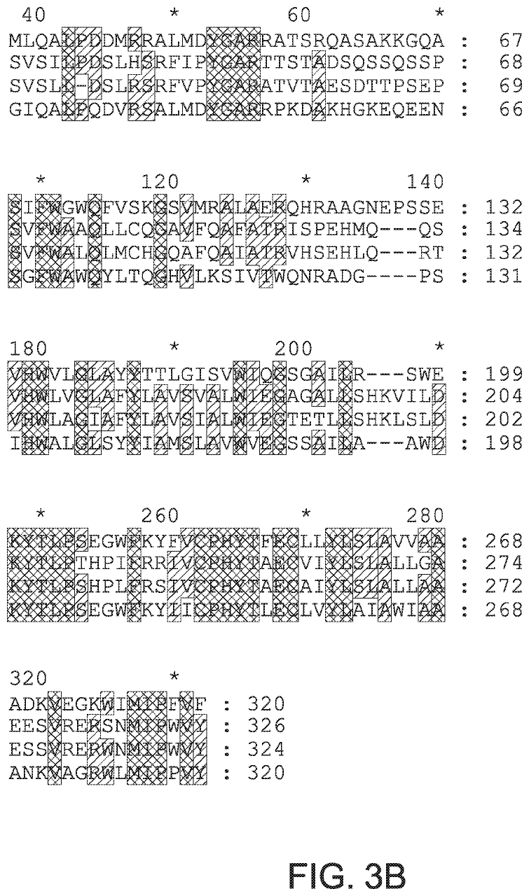

To further investigate the relatedness between the proteins, the proteins were aligned using the MUSCLE algorithm version 3.8.31 with default parameters (Edgar, R. C. (2004). Nucleic Acids Research, 32(5), 1792-1797). The results from this multiple sequence alignment are shown in FIG. 3. The proteins shared significant amino acid sequence identity as indicated in FIG. 3, wherein several stretches/blocks of highly conserved amino acids motifs are shown, such as, the YGAR and/or VPHS[W/Y]F and/or QC[A/V/S]RRL and/or LKKYTLP and/or CPHYT motifs. Together, the results indicate that the putative enzymes likely perform similar functions in the different fungal hosts.

### Example 10: Construction of Plasmid (pNJ00569; SEQ ID NO:27) for Deletion of the TrA1331W Gene (SEQ ID NO:1) Encoding the Putative Steroid Dehydrogenase

A plasmid for deletion of the entire protein encoded by TrA1331W (SEQ ID NO:1) was constructed by cloning a 5′ targeting region, a hph (hygromycin phosphotransferase) and a tk (HSV-1 thymidine kinase) cassette, a repeat to be used for excision of the hph and the tk cassette and a 3′ targeting region into pUC19 (linearized with HindIII and SacI) using an NEBuilder® HiFi DNA Assembly Cloning Kit (New England Biolabs®, Inc.). The 5′ targeting region, the hph and tk cassette, the repeat for excision of the hph and tk cassette and the 3′ targeting region were PCR amplified using the primer sets:

oNJ587 (SEQ ID NO:19)+oNJ588 (SEQ ID NO:28),

oNJ595 (SEQ ID NO:29)+oNJ596 (SEQ ID NO:30),

oNJ592 (SEQ ID NO:31)+oNJ593 (SEQ ID NO:32), and

oNJ589 (SEQ ID NO:33)+oNJ590 (SEQ ID NO:34).

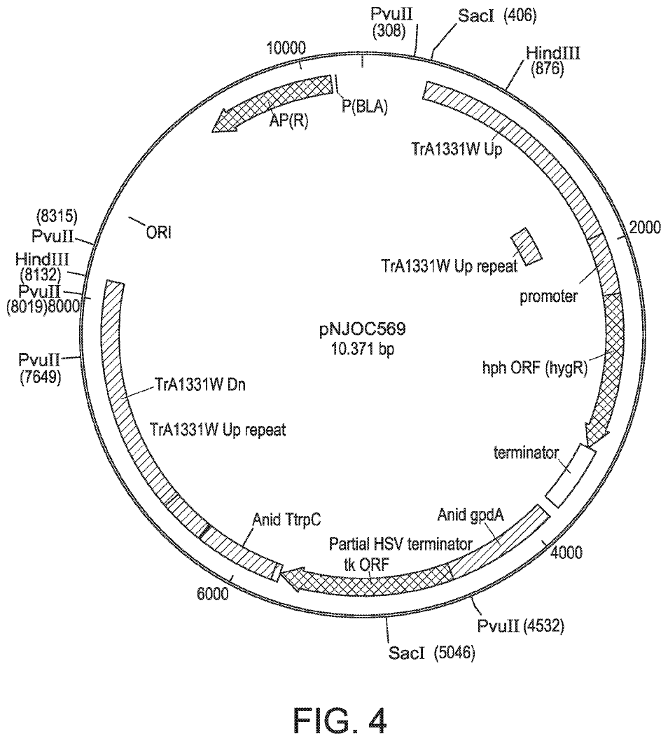

The amplification reactions were performed using Phusion® Hot Start II DNA Polymerase (Thermo Fisher Scientific) according to the manufacturer's instructions. The PCRs were composed of 5 ng of pJfyS1579-41-11 (WO 2010/039840) (template for the hph/tk cassette) or 50 ng of BTR213 genomic DNA (WO 2013/086633) as template, 1×HF buffer, 200 μM of each dNTP, 500 nM forward primer, 500 nM reverse primer, 1 unit of Phusion® Hot Start II DNA Polymerase and sterile Milli-Q® H₂O was added to a final volume of 50 μl. The reactions were incubated in a Bio-Rad C1000 Touch™ Thermal Cycler (Bio-Rad Laboratories) programmed for 1 cycle at 98° C. for 3 minutes; 35 cycles each at 98° C. for 10 seconds, 65° C. for 30 seconds and 72° C. for 30 seconds (the repeat fragment) or 2.5 minutes; and one cycle at 72° C. for 5 minutes. Following thermocycling, the PCR products were separated by 1% agarose gel electrophoresis in TBE buffer and the bands (1587 bp, 4405 bp, 354 bp and 1571 bp) corresponding to the different PCR products were excised from the gel and purified using a NucleoSpin® Gel and PCR Clean-up kit (Macherey-Nagel) according to the manufacturer's instructions. pUC19 was digested with HindIII and SacI in a 50 μl reaction composed of 5 μg pUC19, 20 units each of HindIII-HF (New England Biolabs®, Inc.) and SacI-HF (New England Biolabs®, Inc.), 1× CutSmart® buffer (New England Biolabs®, Inc.) and sterile Milli-Q® H₂O to a final volume of 50 μl. The reaction was incubated at 37° C. and then subjected to 1% agarose gel electrophoresis in TBE buffer. The 2645 bp pUC19 HindIII/SacI fragment was excised from the gel and purified using a NucleoSpin® Gel and PCR Clean-up kit (Macherey-Nagel) according to the manufacturer's instructions. The PCR products and the pUC19 HindIII/SacI fragment were fused together using a NEBuilder® HiFi DNA Assembly Master Mix kit (New England Biolabs) in a total volume of 30 μL composed of 1× NEBuilder® HiFi Assembly Master Mix and 0.04 μmol of each PCR product. The reaction was incubated at 50° C. for 45 minutes and then placed on ice. One μL of the reaction was used to transform 60 μL Stellar™ Competent Cells (Clontech Laboratories, Inc.) according to the manufacturer's instructions. The transformation reaction was spread onto two 2×YT+Amp plates and incubated at 37° C. overnight. Putative transformant colonies were isolated from the selection plates and plasmid DNA was prepared from each one using a QIAprep Spin Miniprep kit (Qiagen) and screened for proper insertion of the fragments by digestion with PvuII. Plasmid DNA giving rise to the expected band pattern (4224 bp, 3117 bp, 2364 bp, 370 bp, 296 bp) upon restriction enzyme digestion was used to create paired-end sequencing libraries and sequenced using 2×150 bp chemistry on a NEXTSEQ™ 500 system (Illumine Inc.). Sequence analysis was performed with the CLC Genomics Workbench version 11.0.1 (QIAGEN). Reads were trimmed using the Trim Reads module. Reads were mapped to a model of the pNJ00569 plasmid (SEQ ID NO:27) using the Map Reads to Reference module with a high-stringency setting. The Basic Variant Detection module was used to detect the presence of any single nucleotide polymorphism. A plasmid having the expected nucleotide sequence was named pNJ00569 (FIG. 4).

### Example 11: Construction of Trichoderma reesei Strain with Deletion of the TrA1331W Gene Encoding the Putative Steroid Dehydrogenase (NJ00584-5D8A)

Trichoderma reesei frt4new-1940-1996-2012-12-1 protoplasts were generated as described in example 2. Approximately 2-4 μg of linearized TrA1331W deletion cassette from pNJ00569 (7716 bp PmeI fragment) was added to 100 μl of protoplast solution and mixed gently. PEG buffer (250 μl) was added, and the reaction was mixed and incubated at 34° C. for 30 minutes. STC (1 ml) was then added and the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days. Next, hygromycin resistant transformants were transferred to PDA plates and incubated at 30° C. for 5-7 days. Transformants were screened for correct integration of the TrA1331 deletion cassette by spore PCR. For each transformant, spores were collected with a sterile 1 μl inoculation loop and suspended in 20 μl of Dilution buffer (PHIRE™ Plant Direct PCR Kit, Thermo Scientific) in a thin-walled PCR tube. Each spore suspension was used as template in a PCR reaction to screen for integration of TrA1331W deletion cassette at the TrA1331W locus. Two PCRs were performed for each transformant; one for the 5′ site of integration and one for the 3′ site of integration. The 5′ integration screen was performed using a primer annealing to a region upstream the site of integration (oNJ632, SEQ ID NO:35) and a primer annealing to a region within the hph and tk cassette (AgJg685, SEQ ID NO:36). The 3′ integration screen was performed using a primer annealing to a region downstream the site of integration (oNJ633, SEQ ID NO:37) and a primer annealing to a region within the hph and tk cassette (AgJg604, SEQ ID NO:38). Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and H₂O to a final volume of 20 μl. Thermocycling was performed according the manufacturer's instructions. The PCR products were analyzed by 1% agarose gel electrophoresis using TBE buffer. Transformants giving rise to the desired PCR products were then subjected to single spore isolation on PDA+1 M sucrose plates. The plates were incubated for 3-5 days at 30° C. Spores from individual colonies were transferred to new PDA plates and the plates were incubated at 30° C. for 5-7 days. The 5′ and 3′ integration verification PCRs described above were repeated and the PCR products were analyzed by 1% agarose gel electrophoresis using TBE buffer. The TrA1331W deletion construct from pNJ00569 contains the hph and HSV-1 tk cassette flanked by direct repeats to facilitate spontaneous loop out of the hph and HSV-1 tk cassette and generation of a clean TrA1331W deletion via homologous recombination between the two repeats. Spores from transformants with correct integration of the TrA1331W modification cassette were collected in H₂O and dilutions were spread onto TrMM plates containing 1.5 μM 5-fluoro-2′-deoxyuridine (FdU) and incubated at 30° C. for 5 days to facilitate identification of isolates having lost the hph and HSV-1 tk cassette. FdU-resistant isolates were then transferred to PDA plates and loss of the hph and HSV-1 tk cassette was verified by spore PCR. In this spore PCR, three primers were added; one primer annealing to a region upstream the hph and tk cassette, one primer annealing to a region outside the 3′ integration site and one primer annealing to a region within the hph and tk cassette. The primers were designed to yield a short or a long PCR product depending upon if the hph and tk cassette was still present. Transformants having lost the hph and tk cassette were then subjected to single spore isolation on PDA+1 M sucrose plates. The plates were incubated for 3-5 days at 30° C. Spores from individual colonies were transferred to new PDA plates and the plates were incubated at 30° C. for 5-7 days. To confirm the presence of the desired deletion of the TrA1331W gene, genomic DNA was prepared for a few isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to a model of the TrA1331W gene (SEQ ID NO:1) using the Map Reads to Reference module with a high-stringency setting. The presence of the desired deletion was verified the InDels and Structural Variants module. One of the isolates containing the desired deletion was named NJ00584-5D8A and saved for further studies.

### Example 12: Transformation of Trichoderma reesei Strain NJ00584-5D8A with pNJOC383

Trichoderma reesei NJ00584-5D8A protoplasts were generated as described in example 2. Approximately 1-10 μg of pNJOC383 (SEQ ID NO:14 and FIG. 2) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC618-81D and saved for further studies.

### Example 13: Lab-Scale Fermentation Showed that Deletion of the Protein Encoded by TrA1331W Also Leads to Increased Lysozyme Productivity/Yield

The four-copy lysozyme expressing strains NJOC587 (control) and NJOC618-81D were evaluated in 2 liter fermentations. Each strain was grown on two PDA plates for 5-7 days at 30° C. Three 500 ml shake flasks, each containing 100 ml of Shake Flask medium, were inoculated with two plugs per shake flask from a PDA plate. The shake flasks were incubated at 28° C. for 48 hours on an orbital shaker at 200 rpm. The cultures were used as seed for fermentation.

A total of 150 ml of each seed culture was used to inoculate 3-liter glass jacketed fermentors (Applikon Biotechnology) containing 1.5 liters of Fermentation Batch medium. The fermentors were maintained at a temperature of 28° C. and pH was controlled using an Applikon 1030 control system to a set-point of 3.5+/−0.1. Air was added to the vessel at a rate of 2.5 L/min and the broth was agitated by Rushton impeller rotating at 300-1100 rpm. Fermentation feed medium composed of autoclaved glucose and phosphoric acid was dosed at a rate of 0 to 14 g/L/hour for a period of approximately 7 days. Aliquots of whole broth were taken on days 5, 6 and 7 and stored at 5 to 10° C. until they were processed for lysozyme activity assay.

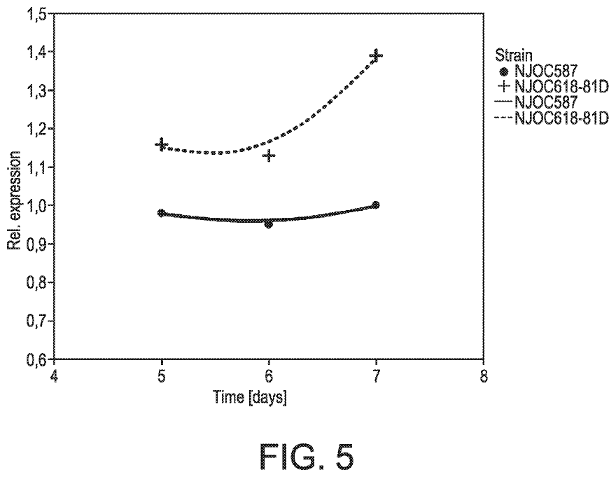

The lysozyme expression level was determined as described in Example 7. Increased lysozyme expression was observed in the steroid dehydrogenase deletion strain NJOC618-81D compared to the NJOC587 (FIG. 5) at all time points assayed. The results demonstrated that inactivation of the TrA1331W gene encoding the native putative steroid dehydrogenase is beneficial for lysozyme expression.

### Example 14: Transformation of Trichoderma reesei Strain frt4new-1940-1996-2012-12-1 with pTmmD-TI_Lipase

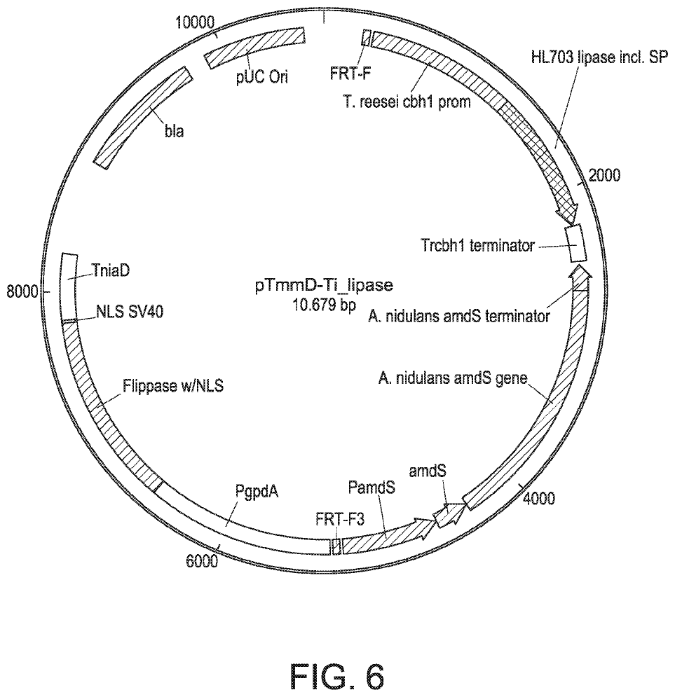

Trichoderma reesei frt4new-1940-1996-2012-12-1 protoplasts were generated as described in example 2. Approximately 1-10 μg of pTmmD-TI_Lipase (Thermomyces lanuginosus HL703 lipase variant expression cassette flanked by FRT-F and FRT-F3 sites for FLP-mediated integration at four loci containing the FRT-F and FRT-F3 sites in the host strain; SEQ ID NO:39 and FIG. 6) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC600-2A and saved for further studies.

### Example 15: Transformation of Trichoderma reesei Strain NJ00586 with pTmmD-TI_Lipase

Trichoderma reesei NJ00586 protoplasts were generated as described in example 2. Approximately 1-10 μg of pTmmD-TI_Lipase (Thermomyces lanuginosus HL703 lipase variant expression cassette flanked by FRT-F and FRT-F3 sites for FLP-mediated integration at four loci containing the FRT-F and FRT-F3 sites in the host strain; SEQ ID NO:39 and FIG. 6) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC609-1A and saved for further studies.

### Example 16: Lipase LU(LXP)/Ml Activity Assay

This method was used in conjunction with a Beckman Coulter Biomek FX and SpectraMax plate reader from Molecular Devices.

Culture supernatants were diluted appropriately in 0.05 M MOPS (3-(N-morpholino)propanesulfonic acid)), 10 mM CaCl₂), 0.01% Triton X-100 buffer pH 7.5 (sample buffer) followed with a series dilution from 0-fold to 1/3-fold to 1/9-fold of the diluted sample. Lipex standard was diluted from 4.0 LU(LXP)/ml concentration and ending with a 0.197 LU(LXP)/ml concentration in the sample buffer. A total of 20 μl of each dilution including standard was transferred to a 96-well flat bottom plate. Two hundred micro-liters of a pNP-palmitate substrate solution (pNPP stock was 7.8 mM pNP-Palmitate in 9.99% EtOH—working solution was: per liter—500 ml 0.1 M MOPS pH 7.5, 20 ml of pNPP stock, 100 ml of 10% Triton X-100, 14.7 ml of 680 mM CaCl₂) and brought up to volume with H₂O) solution was added to each well then incubated at ambient temperature for 30 minutes. During the incubation the rate of the reaction was measured at an optical density of 405 nm for the 96-well plate over a period 20 minutes. Sample concentrations were determined by extrapolation from the generated standard curve.

### Example 17: Lab-Scale Fermentation Showed that Deletion of the Protein Encoded by TrA1331W Also Leads to Increased Lipase Productivity/Yield

The four-copy lysozyme expressing strains NJOC600-2A (control) and NJOC609-1A were evaluated in 2 liter fermentations. Each strain was grown on two PDA plates for 5-7 days at 30° C. Three 500 ml shake flasks, each containing 100 ml of Shake Flask medium, were inoculated with two plugs per shake flask from a PDA plate. The shake flasks were incubated at 28° C. for 48 hours on an orbital shaker at 200 rpm. The cultures were used as seed for fermentation.

A total of 150 ml of each seed culture was used to inoculate 3-liter glass jacketed fermentors (Applikon Biotechnology) containing 1.5 liters of Fermentation Batch medium. The fermentors were maintained at a temperature of 28° C. and pH was controlled using an Applikon 1030 control system to a set-point of 4.5+/−0.1. Air was added to the vessel at a rate of 2.5 L/min and the broth was agitated by Rushton impeller rotating at 300-1100 rpm. Fermentation feed medium composed of autoclaved glucose and phosphoric acid was dosed at a rate of 0 to 15 g/L/hour for a period of approximately five days. Samples (supernatant) were collected on days 2, 3, 4 and 5 and stored at 5° C. until they were processed for lipase activity assay.

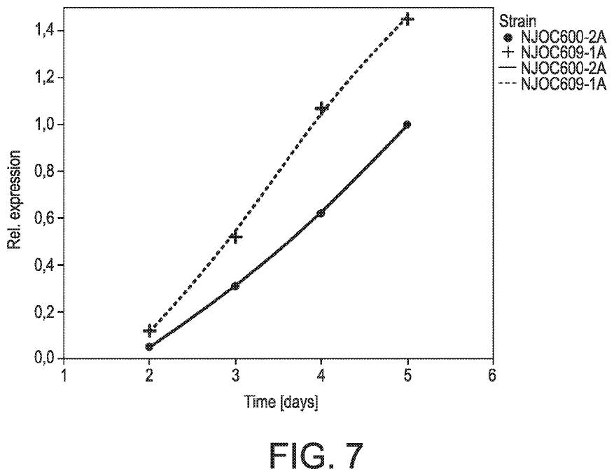

The lipase expression level was determined as described in Example 16. Increased lipase expression was observed in the steroid dehydrogenase deletion strain NJOC609-1A compared to the NJOC600-2A control (FIG. 7) at all time points assayed (ranging between 45-132 improvement). The results demonstrated that inactivation of the TrA1331W gene encoding the native putative steroid dehydrogenase is beneficial for lipase expression.

### Example 18: Transformation of Trichoderma reesei Strain frt4new-1940-1996-2012-12-1 with pSMai326

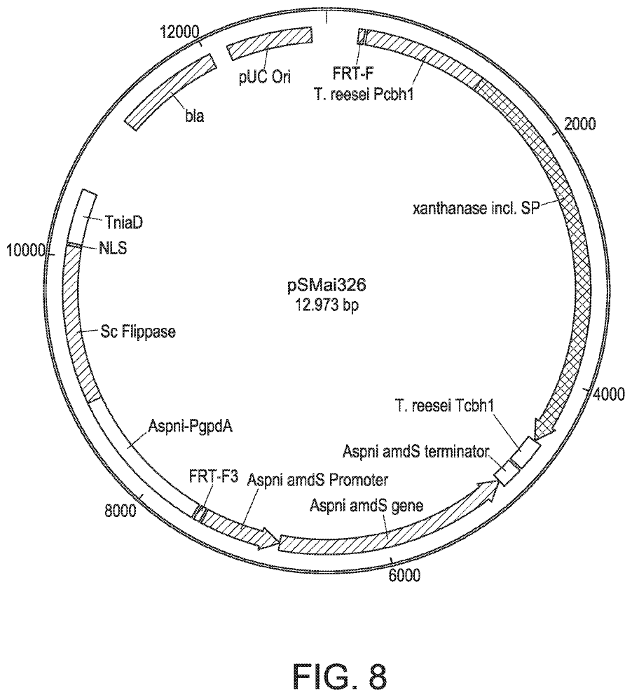

Trichoderma reesei frt4new-1940-1996-2012-12-1 protoplasts were generated as described in example 2. Approximately 1-10 μg of pSMai326 (plasmid containing an Paenibacillus sp. xanthanase variant expression cassette flanked by FRT-F and FRT-F3 sites for FLP-mediated integration at four loci containing the FRT-F and FRT-F3 sites in the host strain; SEQ ID NO:40 and FIG. 8) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC608-1B and saved for further studies.

### Example 19: Transformation of Trichoderma reesei Strain NJ00586 with pSMai326

Trichoderma reesei NJ00586 protoplasts were generated as described in example 2. Approximately 1-10 μg of pSMai326 (plasmid containing an Paenibacillus sp. xanthanase variant expression cassette flanked by FRT-F and FRT-F3 sites for FLP-mediated integration at four loci containing the FRT-F and FRT-F3 sites in the host strain; SEQ ID NO:40 and FIG. 8) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC617-77C and saved for further studies.

### Example 20: Xanthanase XGU(A) Activity Assay

This method was run on a Thermo Arena 30 analyzer. Samples were diluted appropriately with a 0.1 M ACES (N-(2-Acetamido)-2-aminoethanesulfonic acid), 0.056 M NaOH, 4 mM CaCl₂) 2 H₂O, 0.025% Brij® L23 pH 7 buffer (dilution buffer). Dilutions were made to a xanthanase standard to make a 7-point curve using the dilution buffer. Twenty microliters of each sample and standard were added to 125 μl 0.1 M ACES, 0.056 M NaOH (assay buffer) and 50 μl of substrate (0.1% (w/v) xantham gum modified, 0.48% (v/v) ethanol, 0.1 M ACES, 0.06 M NaOH) and incubated at 50° C. for 1200 seconds. One hundred microliters of stopping agent (50 g/L potassium sodium tartrate, 20 g/L PAHBAH (4-Hydroxybenzhydrazide), 5.52 g/L Bismuth (111)-acetate, 0.5 M NaOH) was added to each reaction and incubated at 50° C. for 1200 seconds. An endpoint measurement was made at 405 nm. Sample activities were determined by extrapolation from the generated standard curve.

### Example 21: Lab-Scale Fermentation Showed that Deletion of the Protein Encoded by TrA1331W Also Leads to Increased Xanthanase Productivity/Yield

The four-copy lysozyme expressing strains NJOC608-1B (control) and NJOC617-77C were evaluated in 2 liter fermentations. Each strain was grown on two PDA plates for 5-7 days at 30° C. Three 500 ml shake flasks, each containing 100 ml of Shake Flask medium, were inoculated with two plugs per shake flask from a PDA plate. The shake flasks were incubated at 28° C. for 48 hours on an orbital shaker at 200 rpm. The cultures were used as seed for fermentation.

A total of 150 ml of each seed culture was used to inoculate 3-liter glass jacketed fermentors (Applikon Biotechnology) containing 1.5 liters of Fermentation Batch medium. The fermentors were maintained at a temperature of 28° C. and pH was controlled using an Applikon 1030 control system to a set-point of 4.5+/−0.1. Air was added to the vessel at a rate of 2.5 L/min and the broth was agitated by Rushton impeller rotating at 300-1100 rpm. Fermentation feed medium composed of autoclaved glucose and phosphoric acid was dosed at a rate of 0 to 15 g/L/hour for a period of approximately seven days. Samples (supernatant) were collected on days 2-7 and stored at 5° C. until they were processed for xanthanase activity assay.

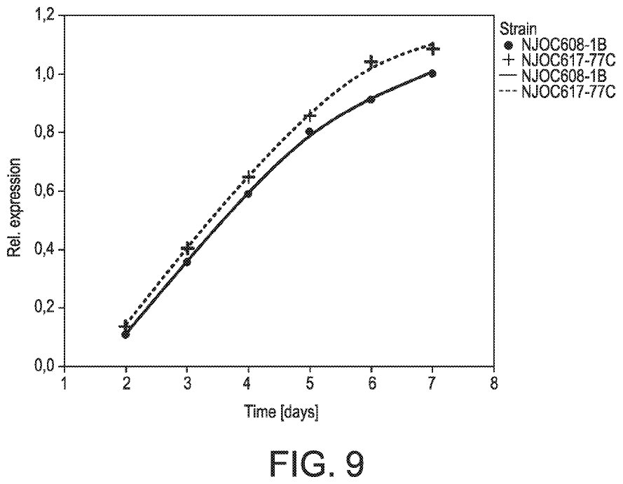

The lipase expression level was determined as described in Example 20. Increased xanthanase expression was observed in the steroid dehydrogenase deletion strain NJOC617-77C compared to the NJOC608-1B control (FIG. 9) at all time points assayed (7-28 improvement). The results demonstrated that inactivation of the TrA1331W gene encoding the native putative steroid dehydrogenase is beneficial for xanthanase expression.

### Example 22: Transformation of Trichoderma reesei Strain frt4new-1940-1996-2012-12-1 with pTmmD-Mf_Lysozyme

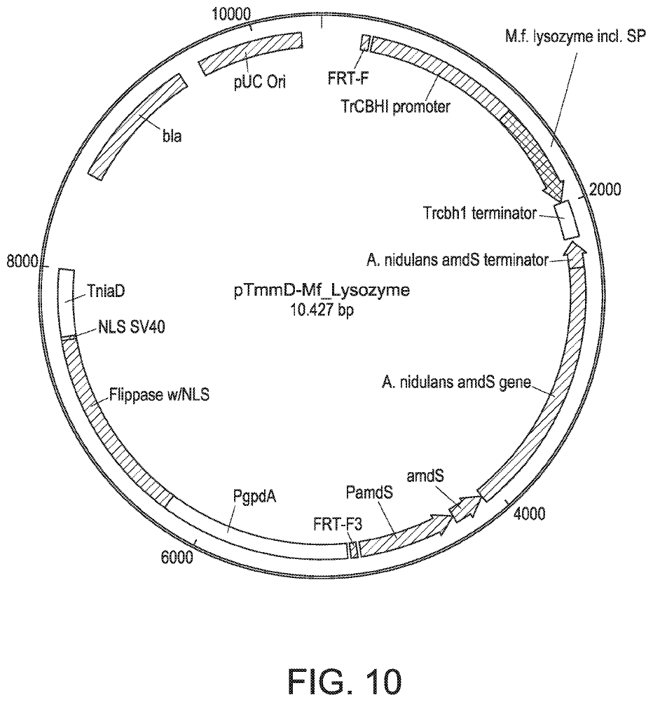

Trichoderma reesei frt4new-1940-1996-2012-12-1 protoplasts were generated as described in example 2. Approximately 1-10 μg of pTmmD-M.f. Lysozyme (plasmid containing an Myceliophthora fergusii lysozyme expression cassette flanked by FRT-F and FRT-F3 sites for FLP-mediated integration at four loci containing the FRT-F and FRT-F3 sites in the host strain; SEQ ID NO:41 and FIG. 10) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC601-5A and saved for further studies.

### Example 23: Transformation of Trichoderma reesei Strain NJ00586 with pTmmD-Mf_Lysozyme

Trichoderma reesei NJ00586 protoplasts were generated as described in Example 2. Approximately 1-10 μg of pTmmD-M.f. Lysozyme (plasmid containing an Myceliophthora fergusii lysozyme expression cassette flanked by FRT-F and FRT-F3 sites for FLP-mediated integration at four loci containing the FRT-F and FRT-F3 sites in the host strain; SEQ ID NO:41 and FIG. 10) was added to 100 μl of the protoplast solution and mixed gently. PEG buffer (250 μl) was added, and the reaction was mixed and incubated at 34° C. for 30 minutes. STC (1 ml) was then added and the contents were spread onto COVE plates for amdS selection. The plates were incubated at 30° C. for 7-9 days. Spores from transformants from each COVE plate were transferred onto COVE2 glucose plates containing 75 μg/ml 5-fluorocytosine (5-FC) (Sigma Chemical Co.) and incubated at 30° C. for 5-7 days. Spores from transformants on the COVE2 glucose plates containing 5-FC were transferred to new COVE2 glucose plates containing 5-FC and incubated at 30° C. for 5-7 days. Several transformants were then subjected to single spore isolation on PDA+1 M sucrose plates. The plates were incubated for 3-5 days at 30° C. Spores from individual colonies were transferred to COVE2 plates and the plates were incubated at 30° C. for 5-7 days. To confirm integration of the lysozyme expression cassette from pNJOC383 at the cbh1, cbh2, eg1 and xyn2 loci, genomic DNA was prepared for a few single spore isolates as described in Example 2 and used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. Reads were mapped to models of the cbh1, cbh2, eg1 and xyn2 loci (SEQ ID NOs:15-18) using the Map Reads to Reference module with a high-stringency setting. One of the isolates having correct integration at all four sites was named NJOC610-2B and saved for further studies.

### Example 24: Lysozyme LSU(A) Activity Assay

This method was used in conjunction with a Beckman Coulter Biomek FX and SpectraMax plate reader from Molecular Devices. Whole broth samples were diluted appropriately in 0.1 M Acetate, 50 mM NaCl, 0.01% Triton X-100 buffer pH 4.5 (sample buffer) followed with a series dilution from 0-fold to 1/3-fold to 1/9-fold of the diluted sample. Lysozyme standard was diluted from 50 LSU(A)/ml concentration and ending with a 2.469 LSU(A)/ml concentration in the sample buffer. A total of 20 μl of each dilution including standard was transferred to a 96-well flat bottom plate. 200 μl of a 0.26 g/L Micrococcus lysodeikticus substrate solution was added to each well then incubated at ambient temperature for 75 minutes. Upon completion of the incubation an optical density of 450 nm was obtained for the 96-well plate. Sample concentrations were determined by extrapolation from the generated standard curve.

### Example 25: Lab-Scale Fermentation Showed that Deletion of the Protein Encoded by TrA1331W Also Leads to Increased M.f. Lysozyme Productivity/Yield

The four-copy lysozyme expressing strains NJOC601-5A (control) and NJOC610-2B were evaluated in 2 liter fermentations. Each strain was grown on two PDA plates for 5-7 days at 30° C. Three 500 ml shake flasks, each containing 100 ml of Shake Flask medium, were inoculated with two plugs per shake flask from a PDA plate. The shake flasks were incubated at 28° C. for 48 hours on an orbital shaker at 200 rpm. The cultures were used as seed for fermentation.

A total of 150 ml of each seed culture was used to inoculate 3-liter glass jacketed fermentors (Applikon Biotechnology) containing 1.5 liters of Fermentation Batch medium. The fermentors were maintained at a temperature of 28° C. and pH was controlled using an Applikon 1030 control system to a set-point of 3.5+/−0.1. Air was added to the vessel at a rate of 2.5 L/min and the broth was agitated by Rushton impeller rotating at 300-1100 rpm. Fermentation feed medium composed of autoclaved glucose and phosphoric acid was dosed at a rate of 0 to 15 g/L/hour for a period of approximately seven days. Aliquots of whole broth were taken on days 4, 5, 6 and 7 and stored at 5 to 10° C. until they were processed for lysozyme activity (LSU(A)) assay.

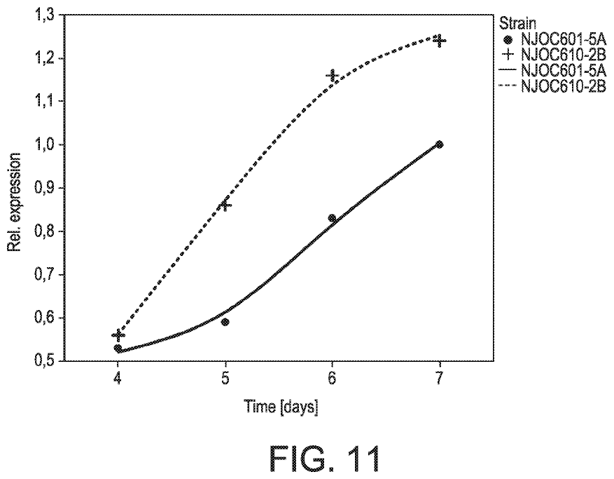

The lipase expression level was determined as described in Example 24. Increased M.f lysozyme expression was observed in the steroid dehydrogenase deletion strain NJOC610-2B compared to the NJOC601-5A control (FIG. 11) at all time points assayed (6-47 improvement). The results demonstrated that inactivation of the TrA1331W gene encoding the native putative steroid dehydrogenase is beneficial for M.f. lysozyme expression.

### EXAMPLES (ASPERGILLUS NIGER)

### Materials and Methods

Unless otherwise stated, DNA manipulations and transformations were performed using standard methods of molecular biology as described in Sambrook et al. (1989) Molecular cloning: A laboratory manual, Cold Spring Harbor lab., Cold Spring Harbor, N.Y.; Ausubel, F. M. et al. (eds.) “Current protocols in Molecular Biology”, John Wiley and Sons, 1995; Harwood, C. R., and Cutting, S. M. (eds.) “Molecular Biological Methods for Bacillus”. John Wiley and Sons, 1990.

Purchased Material (E. coli and Kits)

E. coli DH5a (Toyobo) was used for plasmid construction and amplification. Amplified plasmids are recovered with Qiagen Plasmid Kit (Qiagen). Ligation was done with DNA ligation kit (Takara) or T4 DNA ligase (Boehringer Mannheim). Polymerase Chain Reaction (PCR) was carried out with Expand™ PCR system (Boehringer Mannheim). QIAquick™ Gel Extraction Kit (Qiagen) was used for the purification of PCR fragments and extraction of DNA fragment from agarose gel.

### Enzymes

Enzymes for DNA manipulations (e.g. restriction endonucleases, ligases etc.) are obtainable from New England Biolabs, Inc. and were used according to the manufacturer's instructions.

Plasmids

pBluescript II SK-(Stratagene #212206).

The pHUda963, a derivative of pHUda801 (WO2012/160093), harbouring A. nidulans pyrG gene and herpes simplex virus (HSV) thymidine kinase gene (TK) driven by A. nidulans glyceraldehyde-3-phosphate dehydrogenase promoter (Pgpd) and A. nidulans tryptophane synthase terminator (TtrpC) are described in example 4 in WO2012/160093.

The pJaL1470 harbouring the Acremonium alcalophilus lysozyme (Aa lysozyme) gene is described in WO2015144936A1.

### Microbial Strains

The expression host strains Aspergillus niger C5553 and M1816 (pyrG-phenotype/uridine auxotrophy of C5553) were isolated by Novozymes and are derivatives of Aspergillus niger NN049184 which was isolated from soil described in example 14 in WO2012/160093. C5553 and M1816 have been genetically modified to disrupt expression of amyloglycosidase activities.

### Medium

COVE trace metals solution was composed of 0.04 g of NaB4O7.10H2O, 0.4 g of CuSO4.5H2O, 1.2 g of FeSO4.7H2O, 0.7 g of MnSO4.H2O, 0.8 g of Na2MoO2.2H2O, 10 g of ZnSO4.7H2O, and deionized water to 1 liter.

50× COVE salts solution was composed of 26 g of KCl, 26 g of MgSO4.7H2O, 76 g of KH2PO4, 50 ml of COVE trace metals solution, and deionized water to 1 liter.

COVE medium was composed of 342.3 g of sucrose, 20 ml of 50× COVE salts solution, 10 ml of 1 M acetamide, 10 ml of 1.5 M CsCl2, 25 g of Noble agar, and deionized water to 1 liter.

COVE-N-Gly plates were composed of 218 g of sorbitol, 10 g of glycerol, 2.02 g of KNO3, 50 ml of COVE salts solution, 25 g of Noble agar, and deionized water to 1 liter.

COVE-N (tf) was composed of 342.3 g of sucrose, 3 g of NaNO3, 20 ml of COVE salts solution, 30 g of Noble agar, and deionized water to 1 liter.

COVE-N top agarose was composed of 342.3 g of sucrose, 3 g of NaNO3, 20 ml of COVE salts solution, 10 g of low melt agarose, and deionized water to 1 liter.

COVE-N was composed of 30 g of sucrose, 3 g of NaNO3, 20 ml of COVE salts solution, 30 g of Noble agar, and deionized water to 1 liter.

STC buffer was composed of 0.8 M sorbitol, 25 mM Tris pH 8, and 25 mM CaCl₂).

STPC buffer was composed of 40% PEG 4000 in STC buffer.

LB medium was composed of 10 g of tryptone, 5 g of yeast extract, 5 g of sodium chloride, and deionized water to 1 liter.

LB plus ampicillin plates were composed of 10 g of tryptone, 5 g of yeast extract, 5 g of sodium chloride, 15 g of Bacto agar, ampicillin at 100 μg per ml, and deionized water to 1 liter.

YPG medium was composed of 10 g of yeast extract, 20 g of Bacto peptone, 20 g of glucose, and deionized water to 1 liter.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.

### Transformation of Aspergillus

Transformation of Aspergillus species can be achieved using the general methods for yeast transformation. The preferred procedure for the invention is described below. Aspergillus niger host strain was inoculated to 100 ml of YPG medium supplemented with 10 mM uridine and incubated for 16 hrs at 32° C. at 80 rpm. Pellets were collected and washed with 0.6 M KCl, and resuspended 20 ml 0.6 M KCl containing a commercial β-glucanase product (GLUCANEX™, Novozymes NS, Bagsvrd, Denmark) at a final concentration of 20 mg per ml. The suspension was incubated at 32° C. at 80 rpm until protoplasts were formed, and then washed twice with STC buffer.

The protoplasts were counted with a hematometer and resuspended and adjusted in an 8:2:0.1 solution of STC:STPC:DMSO to a final concentration of 2.5×10⁷ protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove or Cove-N top agarose, the reaction was poured onto Cove or Cove-N (tf) agar plates and the plates were incubated at 30° C. for 5 days.

### PCR Amplifications in Example 1

| Component | Volume | | Final Concentration | |
|---|---|---|---|---|
| 10x Buffer for KOD -Plus- | 5 | μl | 1x | |
| 2 mM dNTPs | 5 | μl | 0.2 mM each | |
| 25 mM MgSO₄ | 2 | μl | 1.0 | mM |
| 10 pmol/μl Primer #1 | 1.5 | μl | 0.3 | μM |
| 10 pmol/μl Primer #2 | 1.5 | μl | 0.3 | μM |
| Template DNA | X μl | | | |
| Genomic DNA Plasmid DNA | 10-200 ng/50 μl | | | |
| | 1-50 ng/50 μl | | | |
| PCR grade water | Y | μl | | |
| KOD-Plus- (1.0 U/μl) | 1 | μl | 1.0 U/50 μl | |
| Total reaction volume | 50 | μl | | |

### 3-Step Cycle:

1. Pre-denaturation: 94° C., 2 min.

2. Denaturation: 94° C., 15 sec.

3. Annealing: Tm-[5-10]° C.*, 30 sec.

4. Extension: 68° C., 1 min./kb

5. Repeat steps #2-4 for a total of 35 cycles.

### Lab-Scale Tank Cultivation for Aa Lysozyme Production

Fermentation was done as fed-batch fermentation (H. Pedersen 2000, Appl Microbiol Biotechnol, 53: 272-277). Selected strains were pre-cultured in liquid media then grown mycelia were transferred to the tanks for further cultivation of enzyme production. Cultivation was done at pH 4.75 at 34° C. for 8 days with the feeding of glucose and ammonium without over-dosing which prevents enzyme production. Culture broth was used for enzyme assay.

### Sequences

SEQ ID NO: 4: Aspergillus niger steroid dehydrogenase genomic DNA sequence
SEQ ID NO: 5: Aspergillus niger steroid dehydrogenase coding sequence (or cDNA)
SEQ ID NO: 6: Aspergillus niger steroid dehydrogenase amino acid sequence

### Example 26 Disruption of the Steroid Dehydrogenase Gene in Aspergillus niger

### Construction of the Steroid Dehydrogenase Gene Disruption Plasmid Plhar473

Plasmid plhar473 was constructed to contain 5′ and 3′ flanking regions for the Aspergillus niger steroid dehydrogenase gene separated by the A. nidulans orotidine-5′-phosphate decarboxylase gene (pyrG) as a selectable marker with its terminator repeats, and the human Herpes simplex virus 1 (HSV-1) thymidine kinase gene. The HSV-1 thymidine kinase gene lies 3′ of the 3′ flanking region of the steroid dehydrogenase gene, allowing for counter-selection of Aspergillus niger transformants that do not correctly target to the steroid dehydrogenase gene locus. The plasmid was constructed in several steps as described below.

A PCR product containing the 3′ flanking region of A. niger steroid dehydrogenase was generated using the following primers:

SEQ ID NO: 42: Primer IH1232-3′steD-F:
5′- aactctctcctctagaTTATGTAGCATGAGACCAGCGGGGA-3′
SEQ ID NO: 43: Primer IH1233-3′steD-R:
5′-acaggagaattcttaattaaAGTCCGGGGTGGGGAGTTTTCA
GGC-3′

The desired fragment was amplified by PCR in a reaction composed of approximately 100 ng of genome DNA of Aspergillus niger NN049184 as described in material and methods. The reaction was incubated in a Bio-Rad® C1000 Touch Thermal Cycler programmed for 1 cycle at 94° C. for 2 minutes; 35 cycles each at 94° C. for 15 seconds, 55° C. for 30 seconds, and 68° C. for 2 minutes; and a 4° C. hold. The resulting 1,500 bp PCR fragment was purified by 0.8 agarose gel electrophoresis using TAE buffer, excised from the gel, and extracted using a QIAQUICK® Gel Extraction Kit.

Plasmid pHUda963 was digested with XbaI and PacI (New England Biolabs Inc.), and purified by 0.8% agarose gel electrophoresis using TAE buffer, where a 8,153 bp fragment was excised from the gel and extracted using a QIAQUICK® Gel Extraction Kit. The 8,153 bp fragment was ligated to the 1,500 bp PCR fragment by using the In-Fusion kit (Clontech Laboratories, Inc.) according to the manufacturer's instructions. One microliter of the reaction mixture was transformed into DH5a chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as plhar473-3′ steD.

A PCR product containing the 5′ flanking region of A. niger steroid dehydrogenase was generated using the following primers:

SEQ ID NO: 44: Primer IH1230-5′steD-F:
5′-gtggcggccgcgtttaaacATCCCTATTTTAAATACCGAGTATG-3′
SEQ ID NO: 45: Primer IH1231-5′steD-R:
5′- tcagtcacccggatccctaATGGTGGCAGTCGTGTTGGATG
CCT-3′

The desired fragment was amplified by PCR in a reaction composed of approximately 100 ng of genome DNA of Aspergillus niger NN049184 as described in material and methods. The reaction was incubated in a Bio-Rad® C1000 Touch Thermal Cycler programmed for 1 cycle at 94° C. for 2 minutes; 35 cycles each at 94° C. for 15 seconds, 55° C. for 30 seconds, and 68° C. for 2 minutes; and a 4° C. hold. The 1,500 bp PCR fragment was purified by 0.8% agarose gel electrophoresis using TAE buffer, excised from the gel, and extracted using a QIAQUICK® Gel Extraction Kit.

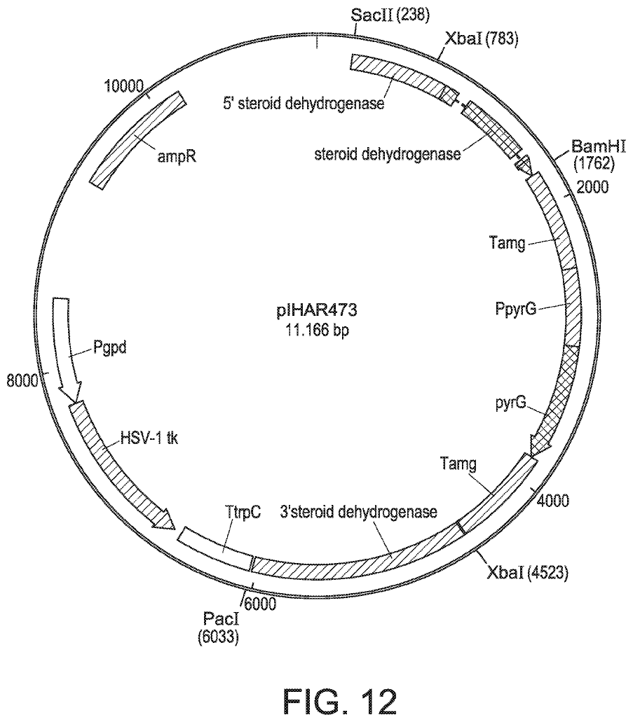

Plasmid plhar473-3′ steD was digested with PmeI and BamHI (New England Biolabs Inc.), and purified by 0.8% agarose gel electrophoresis using TAE buffer, where a 9,653 bp fragment was excised from the gel and extracted using a QIAQUICK® Gel Extraction Kit. The 9,653 bp fragment was ligated to the 1,500 bp PCR fragment by using the In-Fusion kit (Clontech Laboratories, Inc.) according to the manufacturer's instructions. Five μl of the ligation mixture were transformed into DH5a chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as plhar473 (SEQ ID:46, FIG. 12).

Disruption of the Steroid Dehydrogenase Gene in Aspergillus niger Strain M1816

Protoplasts of Aspergillus niger strain M1816 were prepared by cultivating the strain in 100 ml of YPG medium supplemented with 10 mM uridine at 32° C. for 16 hours with gentle agitation at 80 rpm. Pellets were collected and washed with 0.6 M KCl, and resuspended 20 ml 0.6 M KCl containing a commercial β-glucanase product (GLUCANEX™, Novozymes A/S, Bagsvrd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 4 μg of plhar473 was added to 1 ml of the protoplast suspension, mixed gently, and incubated on ice for 30 minutes. Three ml of SPTC was added and the protoplast suspension was incubated for 20 minutes at 37° C. After the addition of 12 ml of 50° C. COVE-N top agarose, the mixture was poured onto the COVE-N plates and the plates were incubated at 30° C. for 7 days. The grown transformants were transferred with sterile toothpicks to Cove-N plates supplemented with 1.5 uM 5-Flouro-2-deoxyuridine (FdU), an agent which kills cells expressing the herpes simplex virus (HSV) thymidine kinase gene (TK) present on plhar473. Single spore isolates were transferred to COVE-N-gly plates.

Possible transformants of Aspergillus niger strain M1816 containing the plhar473 to disrupt the steroid dehydrogenase gene were screened by Southern blot analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) according to the manufacturer's instruction.

Southern blot analysis was performed to confirm the disruption of the steroid dehydrogenase gene locus. Five μg of genomic DNA from each transformant was digested with SpeI. The genomic DNA digestion reactions were composed of 5 μg of genomic DNA, 1 μl of SpeI, 2 μl of 10×NEB CutSmart buffer, and water to 20 μl. Genomic DNA digestions were incubated at 37° C. for approximately 16 hours. The digestions were submitted to 0.8% agarose gel electrophoresis using TAE buffer and blotted onto a hybond N+ (GE Healthcare Life Sciences, Manchester, N.H., USA) using a TURBOBLOTTER® for approximately 1 hour following the manufacturer's recommendations. The membrane was hybridized with a 481 bp digoxigenin-labeled Aspergillus niger steroid dehydrogenase probe, which was synthesized by incorporation of digoxigenin-11-dUTP by PCR using primers IH1252-ste-proF(sense) and IH1253-ste-500R(antisense) shown below:

SEQ ID NO: 47: Primer IH1252-ste-proF:
5′- ATACTCTCCGTCAGCATCCTGCCAG-3′
SEQ ID NO: 48: Primer IH1253-ste-500R:
5′- CTGCTCCTTCGATCCATAAGGCAAC -3′

The amplification reaction (50 μl) was composed of 200 μM PCR DIG Labeling Mix (Roche Applied Science, Palo Alto, Calif., USA), 0.5 μM primers by KOD-Plus (TOYOBO) using plhar473 as template in a final volume of 50 μl. The amplification reaction was incubated in a Bio-Rad® C1000 Touch™ Thermal Cycler programmed for 1 cycle at 94° C. for 2 minutes; 30 cycles each at 94° C. for 15 seconds, 55° C. for 30 seconds, and 68° C. for 30 seconds and a 4° C. hold. PCR products were separated by 0.8% agarose gel electrophoresis using TAE buffer where a 0.5 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker. The strains, 474P2-1 and 474P2-5, giving the correct integration at the steroid dehydrogenase loci (a hybridized band shifted from 5118 by to 7612 bp) were selected for the subsequent experiments.

### Example 27: Expression of the Aa Lysozyme in 474P2-1 and 474P2-5

Chromosomal insertion into A. niger 474P2-1 and 474P2-5 of the Aa lysozyme gene with amdS selective marker (pJaL1470 described in WO2015144936A1) was performed as described in WO 2012/160093. The Aa lysozyme expression plasmids were targeted to four pre-specified loci which are mannosyltransferase (alg2), glucokinase (gukA), acid stable amylase (asaA) and multicopper oxidase (mcoH) by flp recombinase.

Chromosomal insertion into the reference steroid dehydrogenase-wildtype A. niger C5553 of the Aa lysozyme gene was also carried out. Strains which grew well were purified and subjected to southern blotting analysis to confirm whether the Aa lysozyme gene was introduced at mcoH, gukA, asaA and alg2 loci correctly or not. The following set of primers to make non-radioactive probe was used to analyze the selected transformants.

For the promoter region:

SEQ ID NO: 49: HTJP-324 AAGGGATGCAAGACCAAACC
SEQ ID NO: 50: HTJP-325 TGAAGAATTTGTGTTGTCTGAG

Genomic DNA extracted from the selected transformants was digested by SpeI, then probed with the promoter region. By the right gene introduction event, hybridized signals at the size of 6.7 kb (alg2), 2.9 kb (mcoH), 6.7 kb (gukA) and 2.7 kb (asaA) by SpeI and Mlul digestion was observed probed described above.

Among the strains given the right integration events of 4-copies of the genes at mcoH, gukA, asaA and alg2 loci, one strain with the lysozyme from each host (1470-474P2-1, 1470-474P2-5, 1470-05553-13) was selected.

### Example 28: Effect of the Steroid Dehydrogenase Gene Disruption on Enzyme Production

One strain from 474P2-1, 474P2-5 and C5553 was fermented in lab-scale tanks and their enzyme activities (LSU(F)/ml activities) were measured followed by the materials and methods described in Example 7); results are shown in the table below. The steroid dehydrogenase-disrupted strains showed around 1.1 times higher lysozyme (LSU(F)/ml) activity than the reference steroid dehydrogenase-wildtype strain in glass fermenters (Table 3).

TABLE 3
The average LSU(F) activity of the selected three strains
from each host strain, wherein the average LSU(F) yields
from 1470-C5553-13 have been normalized to 1.00.

| | LSU(F) relative |
|---|---|
| Strain | activity |
| A. niger 1470-C5553-13 (wild-type) | 1.00 |
| A. niger 1470-474P2-1 (Δsteroid | 1.09 |
| dehydrogenase) | |
| A. niger 1470-474P2-5 (Δsteroid | 1.08 |
| dehydrogenase) | |

### EXAMPLES (ASPERGILLUS ORYZAE)

### Microbial Strains

The strain AT3091 is expressing the Citrobacter braakii phytase described in WO2006037328 SEQ ID NO.: 4. The strain contains 8 gene copies of the Citrobacter braakii phytase, which have been inserted as tandem inverted repeats at four specific loci on four separate chromosomes using the FLP integration system described in WO2012160093. The host used for creating the strain AT3091 is derived from JaL1903 described in WO2018167153 example 4.

Lab-Scale Tank Cultivation for A. oryzae Strains

Fermentation was done as fed-batch fermentation (H. Pedersen 2000, Appl Microbiol Biotechnol, 53: 272-277) where selected strains were pre-cultured in liquid media then grown mycelia were transferred to the tanks for further cultivation and enzyme production. Cultivation was done for 8 days at 34° C. Ammonia was used for controlling pH. pH was kept at 6 during the batch phase and in the fed bath it was kept at pH 5.4. Feeding was done with maltose syrup. Culture broth was used for enzyme assay.

Phytase activity measurement was performed as described in WO2006037328 Example 4.

### Example 29—Construction and Testing of a Phytase Expression Strain Having the Truncated Steroid Dehydrogenase Gene

For making a truncated steroid dehydrogenase of the A. oryzae steroid dehydrogenase (SEQ ID NO: 9) encoded by SEQ ID NO: 7 (genomic) and SEQ ID NO:8 (cDNA) to resemble the T. reesei steroid dehydrogenase mutant (NJ00586) a stop codon was introduced at amino acid position Y234 by mutating the codon TAT to TAG.

To introduce the stop codon, oligo nucleotide mediated CRISPR gene editing was done as described in Nødvig et al. Fungal Genetics and Biology 115 (2018) 78-89 with the modification that Cas9 was replaced with Mad7 provided from Incripta.

The oligo oAT3303 used for mutation is in the antisense orientation and has the following sequence (SEQ ID NO:51):

TGCTGCGGAACAAGGGGTGGGAGGGAAGGGTGTACTTCTTGAGAGAGTT
AAGCTAGTGGTGGGCGTCATGTTGTAGGCCAGAT.

The two underlined nucleotides introduced mutations in the PAM site to prevent further cutting by the Mad7 complex and the nucleotide in bold changed the codon corresponding to Y234 to a stop codon.

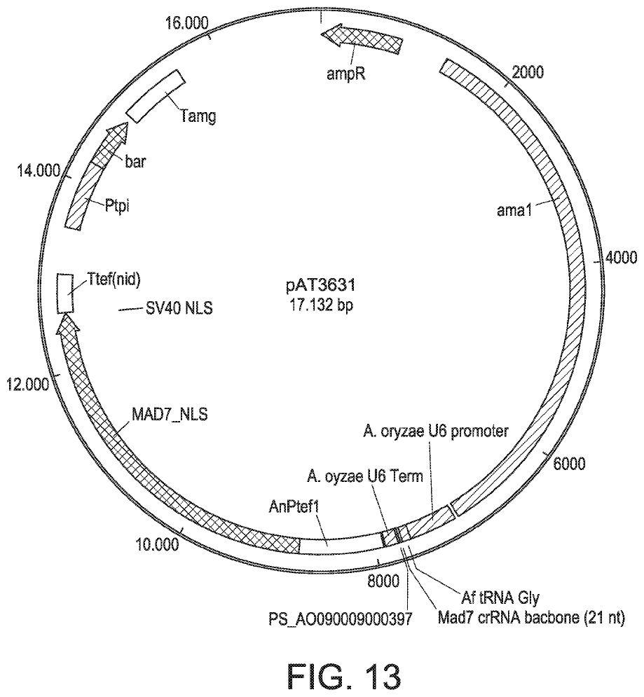

The CRISPR-Mad7 plasmid pAT3631 (FIG. 13, SEQ ID:52) was constructed by modification of pAT1153 (WO19046703, example 25) in the following way: 1) the cas9 gene was exchange with the mad7 gene, 2) the pyrG marker gene was replaced with the bar gene (conferring resistance to bialaphos) (Thompson et al. 1987, EMBO J 6: 2519-2523) and 3) the wA protospacer was replaced with protospacer TCTCTCAAGAAGTACACCCTT (SEQ ID NO:53) targeting the corresponding chromosomal sequence, which has a PAM site TTTC immediately upstream the target sequence.

Aspergillus transformation of AT3091 was performed according to Christensen et al., 1988, Biotechnology 6: 1419-1422. In short, A. oryzae mycelia were grown in a rich nutrient broth. The mycelia were separated from the broth by filtration. The enzyme preparation Glucanex® (Novozymes A/S) was added to the mycelia in an osmotically stabilizing buffer such as 1.2 M MgSO₄ buffered to pH 5.0 with sodium phosphate. The suspension was incubated for 60 minutes at 37° C. with agitation. The protoplasts were filtered through Miracloth® (Calbiochem Inc.) to remove mycelial debris. The protoplasts were harvested and washed twice with STC. The protoplasts were then resuspended in 200-1000 μl of STC.

For transformation of AT3091, 1 μg of plasmid pAT3631 and 100 μmol of repair oligo oAT3303 was added to 100 μl of protoplast suspension and then 200 μl of PEG buffer was added. The mixture was incubated for 20 minutes at room temperature. The protoplasts were harvested, washed twice with 1.2 M sorbitol, and resuspended in 200 μl of 1.2 M sorbitol. Transformants containing the bar gene were selected for their ability to confer resistance to bialaphos when transformants are selected on minimal medium agar plates (Cove, 1966, Biochem. Biophys. Acta. 113: 51-56) containing 1.0 M sucrose as carbon source and 10 mM urea as nitrogen source and 100 mg/l bialaphos. After 5-7 days of growth at 37° C., stable transformants appeared as vigorously growing and sporulating colonies. Transformants were purified once through conidiophores on non-selecting plates (i.e. without bialaphos) whereby the CRISPR-mad7 plasmid was lost.

To verify the expected mutations, PCR was done with the following primers:

(SEQ ID NO: 54)
oAT3163: CTAGCAGTCTCAATCGC
(SEQ ID NO: 55)
oAT3164: TTGACCGTGACAAAGAC

on selected transformants and the resulting 404 bp PCR product was sequenced using the primers as used for PCR. One transformant AT3944 having the stop codon introduced was selected for lab-tank fermentation.

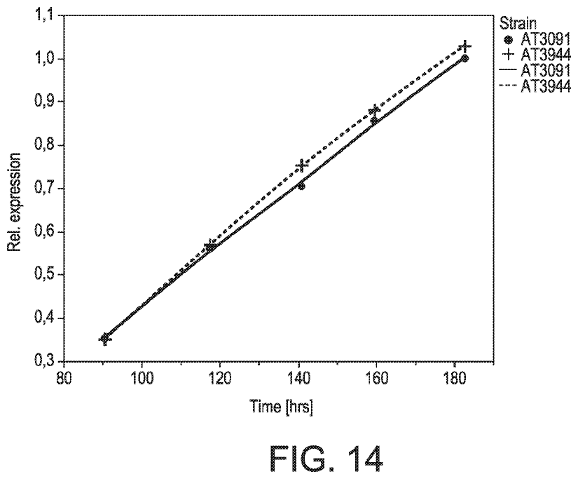

Fermentation of AT3091 and AT3944 and phytase activity was done as described under methods. From 117.4 hrs and until the end of the fermentation, the phytase activity of the AT3944 was 1.4-6.9% higher (ending at −3% higher at 182.7 hrs, FIG. 14). The results indicate that inactivation of the native putative steroid dehydrogenase has a positive impact on phytase expression in Aspergillus oryzae.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-21. (canceled)

22. A mutated filamentous fungal host cell producing a secreted polypeptide of interest,
wherein the mutated host cell comprises a native putative steroid dehydrogenase that is modified, truncated, partly or fully inactivated, present at reduced level or eliminated compared to a non-mutated parent cell,
wherein said native putative steroid dehydrogenase comprises at least one conserved amino acid motif selected from: YGAR, VPHS[W/Y]F, QG[A/V/S]RRL, LKKYTLP and CPHYT; and
wherein the native putative steroid dehydrogenase comprises or consists of an amino acid sequence at least 60% identical to the mature amino acid sequence shown in SEQ ID NO:3, SEQ ID NO:6, SEQ ID NO:9 and/or SEQ ID NO:26.

23. The host cell of claim 22, which is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

24. The host cell of claim 22, which is an Aspergillus aculeatus, Aspergillus aculetinus, Aspergillus awamori, Aspergillus brasiliensis, Aspergillus foetidus, Aspergillus fumigatus, Aspergillus japonicus, Aspergillus luchuensis, Aspergillus nidulans, Aspergillus niger or Aspergillus oryzae.

25. The host cell of claim 22, which is a Trichoderma harzianum, Trichoderma koningii, Trichoderma longibrachiatum, Trichoderma reesei or Trichoderma viride cell.

26. The host cell of claim 22, which is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides or Fusarium venenatum cell.

27. The host cell of claim 22, wherein the secreted polypeptide of interest is an enzyme.

28. The host cell of claim 22, wherein the native putative steroid dehydrogenase is encoded by a gene comprising a nucleotide sequence at least 60% identical to the genomic DNA sequence shown in SEQ ID NO:1, SEQ ID NO:4, SEQ ID NO:7 and/or SEQ ID NO:10.

29. The host cell of claim 22, wherein the native putative steroid dehydrogenase is encoded by a gene comprising a nucleotide sequence at least 60% identical to the cDNA sequence shown in SEQ ID NO:2, SEQ ID NO:5, SEQ ID NO:8 and/or SEQ ID NO:11.

30. The host cell of claim 22, wherein the native putative steroid dehydrogenase is modified, truncated, partly or fully inactivated, present at reduced levels or eliminated compared to a non-mutated parent cell by non-sense or frameshift mutation of the encoding gene, by partial or complete deletion of the encoding gene or by silencing of the encoding gene.

31. A method of producing a mutated filamentous fungal host cell having an improved yield of a secreted polypeptide of interest compared with a non-mutated parent host cell, said method comprising the following steps in no particular order:
a) transforming a filamentous fungal host cell with a polynucleotide construct encoding the secreted polypeptide of interest; and
b) mutating the host cell to modify, truncate, partly or fully inactivate, reduce the level of or eliminate a native putative steroid dehydrogenase, wherein said native putative steroid dehydrogenase comprises at least one conserved amino acid motif selected from YGAR, VPHS[W/Y]F, QG[A/V/S]RRL, LKKYTLP and CPHYT; and wherein the native putative steroid dehydrogenase comprises or consists of an amino acid sequence at least 60% identical to the mature amino acid sequence shown in SEQ ID NO:3, SEQ ID NO:6, SEQ ID NO:9 and/or SEQ ID NO:26.

32. The method of claim 31, wherein the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma.

33. The method of claim 31, wherein the filamentous fungal host cell is an Aspergillus aculeatus, Aspergillus aculetinus, Aspergillus awamori, Aspergillus brasiliensis, Aspergillus foetidus, Aspergillus fumigatus, Aspergillus japonicus, Aspergillus luchuensis, Aspergillus nidulans, Aspergillus niger or Aspergillus oryzae.

34. The method of claim 31, wherein the filamentous fungal host cell is a Trichoderma harzianum, Trichoderma koningii, Trichoderma longibrachiatum, Trichoderma reesei or Trichoderma viride cell.

35. The method of claim 31, wherein the filamentous fungal host cell is a a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides or Fusarium venenatum cell.

36. The method of claim 31, wherein the secreted polypeptide of interest is an enzyme.

37. The method of claim 31, wherein the native putative steroid dehydrogenase is encoded by a gene comprising or consisting of a nucleotide sequence at least 60% identical to the genomic DNA sequence shown in SEQ ID NO:1, SEQ ID NO:4, SEQ ID NO:7 and/or SEQ ID NO:10.

38. The method of claim 31, wherein the native putative steroid dehydrogenase is encoded by a gene comprising or consisting of nucleotide sequence at least 60% identical to the cDNA sequence shown in SEQ ID NO:2, SEQ ID NO:5, SEQ ID NO:8 and/or SEQ ID NO:11.

39. The method of claim 31, wherein the native putative steroid dehydrogenase is modified, truncated, partly or fully inactivated, present at reduced levels or eliminated compared to a non-mutated parent cell by non-sense or frameshift mutation of the encoding gene, by partial or complete deletion of the encoding gene or by silencing of the encoding gene.

40. A method of producing a secreted polypeptide of interest, said method comprising the steps of:
a) cultivating a mutated filamentous fungal host cell according to claim 22 under conditions conducive to the production of the secreted polypeptide; and, optionally,
b) recovering the secreted polypeptide of interest.
